(19) United States
(12) Patent Application Publication
(10) Pub. No.: US 2017/0253927 A1
(43) Pub. Date: Sep. 7, 2017

(54) HERITABLE EPIGENETIC MODIFICATIONS AS MARKERS OF CHEMOTHERAPY EXPOSURE

(71) Applicants: Washington State University, Pullman, WA (US); Seattle Children's Hospital DBA Seattle Children's Research Institute, Seattle, WA (US)

(72) Inventors: Michael K. Skinner, Pullman, WA (US); Margarett Shnorhavorian, Seattle, WA (US)

(21) Appl. No.: 15/446,028

(22) Filed: Mar. 1, 2017

Related U.S. Application Data
(60) Provisional application No. 62/301,651, filed on Mar. 1, 2016.

(51) Int. Cl. C12Q 1/68

(52) U.S. Cl. CPC C12Q 1/6883; C12Q 2600/154

(57) ABSTRACT

Provided herein are epigenetic modifications that are associated with prior exposure to chemotherapy agents. In particular, differential DNA methylation regions (DMRs) that are characteristic of, and can thus be used to identify and/or treat, a male subject who has undergone chemotherapy are provided. The DMRs are used to screen for pregnancy complications, infertility, and passage of heritable mutations to an infant.

### CROSS-REFERENCE TO RELATED APPLICATIONS

This application claims benefit of U.S. provisional patent application 62/301,651, filed Mar. 1, 2016, the complete contents of which is hereby incorporated by reference.

### LENGTHY TABLE

This application includes Table 10 the complete contents of the accompanying text file “Table10.txt”, created Feb. 15, 2017, containing 369 kilobytes, hereby incorporated by reference.

LENGTHY TABLES
The patent application contains a lengthy table section. A copy of the table is available in electronic form from the USPTO web site (http://seqdata.uspto.gov/?pageRequest=docDetail&DocID=US20170253927A1). An electronic copy of the table will also be available from the USPTO upon request and payment of the fee set forth in 37 CFR 1.19(b)(3).

### BACKGROUND OF THE INVENTION

### Field of the Invention

The invention generally relates to the identification of epigenetic modifications that are associated with prior exposure to chemotherapy agents. In particular, the invention provides differential DNA methylation regions (DMRs) that are characteristic of, and can thus be used to identify and/or treat, a male subject who has undergone chemotherapy. The DMRs are used to screen for pregnancy complications, infertility, and passage of heritable mutations to an infant.

### Background of the Invention

The current paradigm for the etiology of heritable diseases, including those caused by environmental insult, is based primarily on mechanisms of genetic alterations such as DNA sequence mutations. However, the majority of inherited diseases have not been linked to specific genetic abnormalities or changes in DNA sequence. In addition, the majority of environmental factors known to cause or influence the development of disease—including heritable diseases—do not have the capacity to alter DNA sequence. Therefore, additional molecular mechanisms need to be taken into account when attempting to clarify the etiology of diseases and to develop diagnostic tools and treatments.

A factor to consider in disease etiology is the importance of early life exposures and events that are critical in later adult onset disease. These developmental origins of disease require a molecular mechanism that does not involve the induction of genetic abnormalities or alterations in DNA sequence. A molecular mechanism that has been shown to mediate the actions of environmental factors on disease is epigenetics. Epigenetics is defined as molecular factors and processes around DNA that regulate genomic activity independent of DNA sequence, and that are mitotically stable. Epigenetic processes include DNA methylation, histone modifications, chromatin structure changes, and some small RNA's.

During migration of the primordial germ cell down the genital ridge the germ cell genome (DNA) becomes demethylated upon colonization of the embryonic gonad. At the onset of gonadal sex determination the germ line then is re-methylated in a sex specific manner. Therefore, the exposure of an environmental factor during this period has the ability to alter the germ line epigenome and if permanently modified can promote a transgenerational phenotype. Therefore, the basic molecular mechanism proposed for environmentally induced epigenetic transgenerational inheritance of adult onset disease involves: 1) environmental exposure during the gonadal sex determination period; 2) alteration in the epigenetic programming (DNA methylation) of the primordial germ cell; 3) permanent alteration in the male germ line epigenome with imprinted-like programming that escapes the de-methylation of DNA at fertilization and during early embryonic development; 4) transmission of the altered sperm epigenome (DNA methylation) to subsequent generations, similar to imprinted-like sites; 5) all cell types and tissues that develop from the sperm have an altered epigenome and transcriptome specific to the cell type or tissue; and 6) increased susceptibility to develop adult onset disease. The transmission of epigenetic information between generations in the absence of any direct environmental exposures is defined as epigenetic transgenerational inheritance.

Advances in chemotherapy-based curative therapy for childhood cancer have led to a significant improvement in outcome, such that long-term survival approaches 80% [1]. This has resulted in an increasing focus on the later life effects of chemotherapy and quality of life in the growing population of survivors of childhood, adolescent and young adult (AYA) cancer. The toxic effect of cancer chemotherapy on reproductive health is one of the most important challenges faced by male childhood and AYA cancer survivors and is a leading cause of decreased quality of life in this population [2-5]. The impact of chemotherapy on subsequent generations has not been previously considered outside the realm of induced genetic mutations. The availability of ancestral environmental epigenetic biomarkers would significantly facilitate the research and development of new screening methods for the identification of subjects whose fertility has been affected by chemotherapy treatment, thus informing future fertility decisions.

### SUMMARY OF THE INVENTION

One aspect of the invention provides a method of determining if a male subject has been exposed to a chemotherapy agent comprising obtaining at least one genomic DNA sequence from a semen sample from said male subject; identifying the presence or absence of an epigenetic modification at one or more regions of said at least one genomic DNA, wherein said epigenetic modification comprises at least one differential DNA methylation region (DMR) listed in Table 6 or Table 10; and determining that said subject has been exposed to a chemotherapy agent if said epigenetic modification is identified to be present in said at least one genomic DNA sequence.

In some embodiments, the epigenetic modification comprises a plurality of DMRs selected from the group listed in Table 6 or Table 10. In other embodiments, the epigenetic modification comprises each DMR listed in Table 6 or Table 10. In some embodiments, the chemotherapy agent is at least one of cisplatin and ifosfamide.

Another aspect of the invention provides a method of screening for pregnancy complications, infertility, and passage of heritable mutations to an infant attributable to a male subject that has previously undergone chemotherapy treatment comprising obtaining at least one genomic DNA sequence from a semen sample from said male subject that has previously undergone chemotherapy treatment; identifying the presence or absence of an epigenetic modification at one or more regions of said at least one genomic DNA, wherein said epigenetic modification comprises at least one DMR listed in Table 6 or Table 10; and indicating that said subject is at high risk of infertility or of passing heritable mutations which can lead to pregnancy complications or mutations in an infant if said epigenetic modification is identified to be present in said at least one genomic DNA sequence.

In some embodiments, the epigenetic modification comprises a plurality of DMRs selected from the group listed in Table 6 or Table 10. In other embodiments, the epigenetic modification comprises each DMR listed in Table 6 or Table 10. In some embodiments, the male subject underwent chemotherapy treatment for a period of time at an age prior to reproduction. In some embodiments, the male subject underwent chemotherapy treatment for a period of time at an age from 14 and 20 years old. In some embodiments, the chemotherapy treatment comprised at least one of cisplatin and ifosfamide.

A further aspect of the invention provides a method for the early intervention and treatment of a male subject who is suspected of or who has been exposed to chemotherapy treatment, comprising obtaining at least one genomic DNA sequence from a semen sample from said male subject that has previously undergone chemotherapy treatment; identifying the presence or absence of an epigenetic modification at one or more regions of said at least one genomic DNA, wherein said epigenetic modification comprises at least one DMR listed in Table 6; indicating that said subject is at high risk of infertility or of passing heritable mutations which can lead to pregnancy complications or mutations in an infant if said epigenetic modification is identified to be present in said at least one genomic DNA sequence; and administering an appropriate treatment protocol to said subject determined to be at high risk of infertility or of passing heritable mutations which can lead to pregnancy complications or mutations in an infant.

### BRIEF DESCRIPTION OF THE DRAWINGS

FIG. 1. Human sperm chemotherapy-associated DMR chromosome location. The DMR locations on the individual chromosomes are presented as an arrowhead. Only DMR containing at least two significant sites at a p-value threshold of 1e-04 are shown. The box under the chromosome line represents statistically significant overrepresented clusters of DMR within the chromosomal size of the box.

FIG. 2. The human sperm chemotherapy-associated all site DMR locations on the individual chromosomes is presented. All site (single and multiple site) DMR are represented with an arrowhead and the DMR clusters with a box. All site DMRs at a p-value threshold of 1e-04 are shown.

FIG. 3A-B. Human sperm chemotherapy-associated DMR genomic feature and size. A) The CpG density of the DMR is presented as number of CpG/100bp with the corresponding number of DMR. B) The DMR length in kilobase pairs (kb) is presented with the corresponding DMR number. Only DMR containing multiple significant windows at a p-value threshold of 1e-04 are shown.

FIG. 4. The human sperm chemotherapy-associated DMR associated gene classifications (i.e. functional categories). The number of DMR associated genes for specific classification categories are presented.

### DETAILED DESCRIPTION

Epigenetic transgenerational inheritance provides an alternative molecular mechanism for germ line transmission of environmentally induced phenotypic change compared to that of classic genetics. Most factors do not have the ability to modify DNA sequence, but environmental factors such as nutrition or various toxicants can influence epigenetic processes to mediate alterations in genome activity. Environmental epigenetics focuses on how a cell or organism responds to environmental factors or insults to create altered phenotypes or disease. Until the present invention, it was previously unknown how chemotherapy or radiotherapy treatment affected the epigenetic programming of a germ line and whether such impact could influence later life fertility and epigenetic inheritance.

Described herein are the altered DNA methylation profiles in the germ line (sperm) of male subjects after exposure to chemotherapy agents. In particular, Table 6 provides statistically significant epimutations, termed DMRs, that were identified in the germ line of male subjects who had undergone chemotherapy treatment. Due to the imprinted-like nature of the altered epigenetic DNA methylation sites, the germ line (sperm) transmit this epigenome phenotype to subsequent generations, which is termed epigenetic transgenerational inheritance. Without being bound by theory, the basic mechanism involves the ability of an environmental factor, such as a chemotherapeutic agent, to alter the germ line DNA methylation program to promote imprinted-like sites that then transmit an altered epigenome phenotype transgenerationally. In some cases, environmental exposures act on somatic cells at critical windows of development to influence phenotype and/or disease in the individual exposed, but this will not become transgenerational. In the event the critical window for the primordial germ cell is affected by environmental exposure, the altered germ line has the ability to promote a transgenerational phenotype for subsequent generations.

Epigenetic regulatory sites and epigenetic mutation sites (such as those involving differential DNA methylation) have profound regulatory effects on gene expression, cell function and the development of abnormal physiology and disease. The presence of such sites in the germline (e.g. sperm) can promote epigenetic transgenerational inheritance of, e.g. adult onset disease. Therefore, identification of these epimutations and/or epigenetic control regions (referred to collectively herein as “epigenetic control regions” or “ECRs”) is critical to understanding disease etiology and heritable conditions that do not follow classic Mendelian genetics, and to the diagnosis and treatment of such conditions.

Provided herein are DMRs which are useful for the identification of subjects who have undergone chemotherapy or radiotherapy treatment. In some embodiments, the methylation level is determined by a cytosine. In some embodiments, the DMRs are associated with certain genes in an individual. In some embodiments, the DMRs are associated with certain CpG loci. The CpG loci may be located in the promoter region of a gene, in an intron or exon of a gene or located near the gene in a patient's genomic DNA. In an alternate embodiment, the CpG may not be associated with any known gene or may be located in an intergenic region of a chromosome. In some embodiments, the CpG loci may be associated with one or more than one gene.

In some instances, the DMRs described herein are found in CpG desert regions of the genome, e.g. a CpG density of about 10% or less or a mean around two CpG per 100 base pairs. Due to the evolutionary conservations of CpG clusters in a CpG desert, these are likely epigenetic regulatory sites. Additional genomic features of characteristic of ECRs are described in U.S. Patent Publication 2013/0226468 incorporated herein by reference. Those of skill in the art will recognize that the “%” of a sequence of interest (e.g. CpG) means that the sequence occurs the indicated number of times per 100 base pairs analyzed, e.g. 15% or less CpG means that the dinucleotide sequence C followed by G occurs at most 15 times per 100 base pairs within a DNA segment that is analyzed. Analyses are usually carried out by iterative analysis of consecutively overlapping sequences within a large DNA molecule of interest, e.g. a chromosome, a section of a chromosome, etc.

The DMRs provided herein allow for determining if a male subject has been exposed to a chemotherapy agent comprising obtaining at least one genomic DNA sequence from a semen sample from said male subject; identifying the presence or absence of an epigenetic modification at one or more regions of said at least one genomic DNA, wherein said epigenetic modification comprises at least one differential DNA methylation region (DMR) listed in Table 6; and determining that said subject has been exposed to a chemotherapy agent if said epigenetic modification is identified to be present in said at least one genomic DNA sequence.

In some embodiments, the epigenetic modification comprises a plurality of DMRs selected from the group listed in Table 6. In other embodiments, the epigenetic modification comprises all 135 DMRs listed in Table 6. In some embodiments, the epigenetic modification consists of all 135 DMRs listed in Table 6.In some embodiments, the chemotherapy agent is at least one of cisplatin and ifosfamide.

Contemplated herein is the use of one or more DMRs listed in Table 10. Table 10 includes human sperm DMR for all DMR sites, single and multiple, at a p-value threshold of 1e-04. The DMR name, chromosome location, start and stop base pair location, length in base pair (bp), number of significant windows (100 bp), p-value, number of CpG sites, CpG sites per 100 bp, and DMR associated gene symbol (annotation) are provided.

A “plurality” as used herein refers to two or more DMRs, for example, two, three, four, five, six, and every integer up to and including all 135 DMRs listed in Table 6. A plurality may also refer to two or more DMRs listed in Table 10 and every integer up to and including all DMRs listed in Table 10.

Another aspect of the invention provides a method of screening for pregnancy complications, infertility, and passage of heritable mutations to an infant attributable to a male subject that has previously undergone chemotherapy treatment comprising obtaining at least one genomic DNA sequence from a semen sample from said male subject that has previously undergone chemotherapy treatment; identifying the presence or absence of an epigenetic modification at one or more regions of said at least one genomic DNA, wherein said epigenetic modification comprises at least one DMR listed in Table 6 or Table 10; and indicating that said subject is at high risk of infertility or of passing heritable mutations which can lead to pregnancy complications or mutations in an infant if said epigenetic modification is identified to be present in said at least one genomic DNA sequence.

In some embodiments, the epigenetic modification comprises a plurality of DMRs selected from the group listed in Table 6 or Table 10. In other embodiments, the epigenetic modification comprises each DMR listed in Table 6 or Table 10. In some embodiments, the male subject underwent chemotherapy treatment for a period of time at an age prior to reproduction. In some embodiments, the male subject underwent chemotherapy treatment for a period of time at an age from 14 and 20 years old, e.g. at 14, 15, 16, 17, 18, 19, and 20 years old. In some embodiments, the subject was under 14 years old or above 20 years old at the time of treatment. In some embodiments, the chemotherapy treatment comprised at least one of cisplatin and ifosfamide.

“Epimutation” and “epigenetic modification” as used herein refer to modifications of cellular DNA that affect gene expression without altering the DNA sequence. The epigenetic modifications are both mitotically and meiotically stable, i.e. after the DNA in a cell (or cells) of an organism has been epigenetically modified, the pattern of modification persists throughout the lifetime of the cell and is passed to progeny cells via both mitosis and meiosis. Therefore, with the organism's lifetime, the pattern of DNA modification and consequences thereof, remain consistent in all cells derived from the parental cell that was originally modified. Further, if the epigenetically modified cell undergoes meiosis to generate gametes (e.g. sperm), the pattern of epigenetic modification is retained in the gametes and thus inherited by offspring. In other words, the patterns of epigenetic DNA modification are transgenerationally transmissible or inheritable, even though the DNA nucleotide sequence per se has not been altered or mutated. Without being bound by theory, it is believed that enzymes known as methyltransferases shepherd or guide the DNA through the various phases of mitosis or meiosis, reproducing epigenetic modification patterns on new DNA strands as the DNA is replicated. Exemplary epigenetic modifications include, but are not limited, to DNA methylation, histone modifications, chromatin structure modifications, and non-coding RNA modifications, etc.

Epigenetic modifications may be caused by exposure to any of a variety of factors, examples of which include but are not limited to: chemical compounds e.g. endocrine disruptors such as vinclozolin; chemicals such as those used in the manufacture of plastics e.g. bispheol A (BPA); bis(2-ethylhexyl)phthalate (DEHP); dibutyl phthalate (DBP); insect repellants such as N, N-diethyl-meta-toluamide (DEET); pyrethroids such as permethrin; various polychlorinated dibenzodioxins, known as PCDDs or dioxins e.g. 2,3,7,8-tetrachlorodibenzo-p-dioxin (TCDD); extreme conditions such as abnormal nutrition, starvation, etc. In preferred embodiments, the subject of the invention has been exposed to one or more chemotherapeutic agents which include alkylating agents such as ifosfamide and cyclophosphamide, anthracyclines such as daunorubicin and doxorubicine, taxanes such as paclitaxel and docetaxel, epothilones, histone deacetylase inhibitors, topoisomerase inhibitors, kinase inhibitors such as gefitinib, platinum-based agents such as cisplatin, retinoids, and vinca alkaloids, etc. These agents may be used to treat a variety of cancers, including but not limited to, an osteosarcoma, lymphoma, melanoma, etc.

Methods of identifying DMRs in genomic DNA are well known to one skilled in the art. For example, microarray based methylome profiling and bioinformatics data analysis may be used to analyze DNA methylation profiles. In some embodiments, the microarray chip is a tiling array chip. In some embodiments, Methylated DNA immunoprecipitation (MeDIP) followed by next generation sequencing (NGS) is used. In some embodiments, MeDIP-Chip is used. Additional methods for detecting methylation levels can involve genomic sequencing before and after treatment of the DNA with bisulfite. When sodium bisulfite is contacted to DNA, unmethylated cytosine is converted to uracil, while methylated cytosine is not modified. Bisulfite methods may also be used in conjunction with pyrosequencing and PCR. Computer executable algorithms and software programs for implementing the same are also encompassed by the invention. Such software programs generally contain instructions for causing a computer to carry out the steps of the methods disclosed herein. The computer program will be embedded in a non-transient medium such as a hard drive, DVD, CD, thumb drive, etc.

The invention also provides kits for the detection and/or quantification of the epigenetic modification described herein using the methods described herein. In some embodiments, the kit comprises at least one polynucleotide that hybridizes to one of the DMR loci identified in Table 6 or Table 10 (or a nucleic acid sequence at least 90% identical to the DMR loci of Table 6 or Table 10), or that hybridizes to a region of DNA flanking one of the DMR loci identified in Table 6 or Table 10, and at least one reagent for detection of gene methylation, Reagents for detection of methylation include, e.g., sodium bisulfite, polynucleotides designed to hybridize to sequence at or near the DMR loci of the invention if the sequence is not methylated, and/or a methylation-sensitive or methylation-dependent restriction enzyme. The kits can provide solid supports in the form of an assay apparatus that is adapted to use in the assay. The kit may further comprise detectable labels, optionally linked to a polynucleotide, e.g., a probe, in the kit. Other materials useful in the performance of the assays can also be included in the kit, including test tubes, transfer pipettes, and the like. The kit can also include written instructions for the use of one or more of these reagents in any of the assays described herein.

Selection and identification of a subject for analysis may be predicated on and/or influenced by any number of factors. For example, the subject or subjects may be known or suspected to be afflicted with a disease or condition associated with epigenetic mutations; or who have been or are suspected of having been exposed to an agent that causes, or is suspected of causing, epigenetic mutations; or who have inexplicably inherited a disease or disease condition from a parent for which no DNA sequence mutation has been identified, etc. Subjects whose DNA is analyzed may be of any age, and in any stage of development, so long as cells containing a DNA sequence of interest can be obtained from the subject. For example, the subject may be an adult, an adolescent, a laboratory animal, etc. The cells from which the DNA is obtained may be any suitable cell, including but not limited to gametes, cells from swabs such as buccal swabs, cells sloughed into amniotic fluid, etc.

The genomic features described herein may be used in a variety of applications. For example, the DMRs of the invention can be indicative of having, the risk of having, or the risk of developing infertility or a condition that could lead to pregnancy complications and/or passage of heritable mutations to an infant. Thus the methods of the invention may be used, for example, in an in vitro fertilization clinic setting to test sperm for epimutations and for the potential to pass epigenetic information to offspring. The methods of the invention are also useful for screening potential sperm donors at a donation center. Further applications include screening applicants for health insurance coverage.

The DMRs of the invention can serve as biomarkers to be used e.g. in disease diagnosis and/or to detect environmental exposures to agents or conditions that cause epimutations and/or to monitor therapeutic responsiveness to a medicament or treatment and/or used as prognostic indicators. The detection of epigenetic modifications at the regions described herein (i.e. a positive diagnostic result) will suggest or confirm that the subject has, indeed, likely been exposed to chemotherapy and/or radiotherapy treatments, and treatments suitable for said exposure, or the effects of said exposure, can be instituted. For example, chemotherapy exposure may result in a low sperm count in the male patient leading to infertility. Thus, an appropriate infertility treatment, such as surgical extraction of sperm, may be implemented. In some instances, a male subject may cryopreserve a sperm sample before or shortly after undergoing chemotherapy and/or radiotherapy treatment. In other instances, a male subject may decide to utilize a sperm donor due to the subject's infertility or to prevent the possibility of pregnancy complications and/or the passage of heritable mutations to an infant attributable to the male subject.

Information concerning the type and extent of epigenetic modification in a subject may be used in a variety of decision making processes undertaken by a subject that is tested. For example, depending on the severity of the symptoms caused by an epigenetic modification that is identified, a subject may decide to forego having children or to terminate a pregnancy in order to prevent transmission of the modification to offspring. Diagnostic tests based on the present invention can be included in prenatal testing.

Thus, an aspect of the invention provides a method for the early intervention and treatment of a male subject who is suspected of or who has been exposed to chemotherapy treatment, comprising obtaining at least one genomic DNA sequence from a semen sample from said male subject that has previously undergone chemotherapy treatment; identifying the presence or absence of an epigenetic modification at one or more regions of said at least one genomic DNA, wherein said epigenetic modification comprises at least one DMR listed in Table 6 or Table 10; indicating that said subject is at high risk of infertility or of passing heritable mutations which can lead to pregnancy complications or mutations in an infant if said epigenetic modification is identified to be present in said at least one genomic DNA sequence; and administering an appropriate treatment protocol to said subject determined to be at high risk of infertility or of passing heritable mutations which can lead to pregnancy complications or mutations in an infant.

In contrast, a negative result (no epigenetic modification at the site) suggests that the subject has not been exposed to chemotherapy and/or radiotherapy treatments (or at least that the exposure did not result in damage). If it is known that exposure did occur, then prophylactic screening of a DNA sample from a patient can result in early identification of a risk of disease and lead to early therapeutic intervention. In addition, ongoing monitoring of the extent of epigenetic modification of a site can provide valuable information regarding the outcome of the administration of agents (e.g. drugs or other therapies) which are intended to treat or prevent a condition caused by epimutation, i.e. the therapeutic responsiveness of a patient. Those of skill in the art will recognize that such analyses are generally carried out by comparing the results obtained using an unknown or experimental sample with results obtained a using suitable negative or positive controls, or both.

Subjects whose DNA is analyzed may be suffering from any of a variety of disorders (diseases, conditions, etc.) including but not limited to: various known late or adult onset conditions, such as low sperm production, infertility, abnormalities of sexual organs, kidney abnormalities, prostate disease, immune abnormalities, behavioral effects, etc. In other embodiments, no symptoms are present but screening using the diagnostics is employed to rule out the presence of “silent” epigenetic mutations which could cause disease symptoms in the future, or which could be inherited and cause deleterious effects in offspring.

The DMRs described herein may also be used to screen and identify therapeutic modalities for the treatment of epigenetic mutations due to chemotherapy and/or radiotherapy exposure. Those of skill in the art will recognize that such methods of screening are typically carried out in vitro, e.g. using a DNA sequence that is immobilized in a vessel, or that is present in a cell. However, such tests may also be carried out in model laboratory animals. In one embodiment, candidate agents which reverse epigenetic modification are screened by analyzing the regions. In another embodiment, candidate agents which prevent epigenetic modifications are screened by analyzing the regions. In this way, the epigenetic biomarkers described herein can be used to facilitate, e.g. drug development and clinical trials patient stratification (i.e. pharmacoepigenomics).

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting example which further illustrates the invention, and is not intended, nor should it be interpreted to, limit the scope of the invention.

### EXAMPLE

### Differential DNA Methylation Regions in Adult Human Sperm following Adolescent Chemotherapy

### Abstract

The potential that adolescent chemotherapy can impact the epigenetic programming of the germ line to influence later life adult fertility and promote epigenetic inheritance was investigated. Adult males approximately ten years after pubertal exposure to chemotherapy were compared to adult males with no previous exposure. Sperm were collected to examine differential DNA methylation regions (DMR) between the exposed and control populations. A signature of statistically significant DMRs was identified in the chemotherapy exposed male sperm. The DMRs, termed epimutations, were found in CpG desert regions of primarily 1 kilobase size. Gene associations and correlations to genetic mutations (copy number variation) were also investigated. Observations indicate adolescent chemotherapy exposure can promote epigenetic alterations that persist in later life. This is the first observation in humans that an early life chemical exposure can permanently reprogram the spermatogenic stem cell epigenome. The germline (i.e. sperm) epimutations identified suggest chemotherapy can promote epigenetic inheritance to the next generation.

### Introduction

Previous studies have demonstrated transient early life toxicant exposures can influence later life health effects and epigenetic reprogramming of the germline in animal models [6-8]. Epigenetics is defined as “molecular factors or processes around DNA that regulate genome activity independent of DNA sequence and are mitotically stable” [6, 9]. The currently known epigenetic mechanisms include DNA methylation, histone modifications, selected non-coding RNA and chromatin structure [6]. Although the vast majority of environmental factors can not alter DNA sequence, most have the ability to alter epigenetic programming during development [6, 9]. Early developmental exposures have been shown to alter the epigenetic programming of cells associated with a number of adult onset diseases [6, 9-11]. Environmentally-induced DNA methylation changes in Sertoli or granulosa cells have been shown to associate with testis and ovarian disease in the adult [12, 13]. Environmental epigenetics provides a molecular mechanism for the developmental origins of disease [9]. In the event the altered epigenetic programming occurs in the germline (sperm or egg), the altered epigenetics (e.g. epimutations) have the potential to be transmitted between generations [6-8, 14]. A number of studies have demonstrated that environmental factors (e.g. toxicants and nutrients) following fetal exposure can alter the germline epigenome (e.g. DNA methylation) to then transmit epimutations to subsequent generations [8, 14].

When the germline transmission of epigenetic information occurs between multiple generations in the absence of continuous exposure this is considered to be environmentally-induced epigenetic transgenerational inheritance [6, 7]. This form of non-genetic inheritance is due to the germline transmission of epigenetic information. A number of studies have shown that numerous environmental toxicants such as fungicides [7], plastics [15], pesticides [7] and hydrocarbons [16] can promote the epigenetic transgenerational inheritance of disease [6]. The transgenerational disease observed includes testis, ovary, prostate, mammary, kidney and brain disease [17, 18]. The majority of these transgenerational studies have observed correlations between the phenotypes and differential DNA methylation alterations in the sperm [14]. Therefore, early life environmental exposures can influence the epigenetic programming of the sperm and have the ability to promote epigenetic inheritance to subsequent generations.

The generational impact of chemotherapy has not been thoroughly investigated. Therefore, the current study was designed to investigate the actions of chemotherapy on pubertal males that potentially promote an alteration in epigenetic programming that will result in adult male sperm having epimutations. This requires the spermatogonial stem cell population in the testis to be affected permanently to produce later life effects on the sperm epigenome. Osteosarcoma is one of the most common cancers in this population treated with agents such as cisplatin and ifosfamide. This population provides a useful model to investigate potential chemotherapy induced effects on later life reproductive health. Previous studies have demonstrated altered DNA methylation profiles in control versus infertile human male sperm [19]. The presence of sperm epimutations due to adolescent chemotherapy also would suggest for the first time the potential for epigenetic inheritance to the next generation.

### Results

Characteristics of the chemotherapy-exposed patients and controls including age of semen collection, specific chemotherapy and sperm quality are presented in Tables 1 and 2. The age of exposure ranged 14 to 20 years and the chemotherapy was cisplatin with some also including ifosfamide. Upon collection the sperm numbers ranged from 7 to 518 million total with the control population mean of 280 million total per individual and the chemotherapy-exposed mean of 77.8 million total per individual. Therefore, there was a general reduction in sperm number in the chemotherapy-exposed population, as previously described [2, 3].

TABLE 1
Patient Information. Information for the chemotherapy treated
and control individuals with treatment age, sperm collection age,
chemotherapy used and total sperm number presented.

| | | Collection | Sperm # | |
|---|---|---|---|---|
| ID # | Chemotherapy | Age (yr) | (Total Millions) | Pool # |
| CIS-051 | N/A | 40 | 240.5 | 1 |
| CIS-056 | N/A | 38 | 303.6 | 1 |
| CIS-061 | N/A | 32 | 44.2 | 1 |
| CIS-063 | N/A | 33 | 675 | 2 |
| CIS-067 | N/A | 39 | 262 | 2 |
| CIS-073 | N/A | 37 | 280.4 | 2 |
| CIS-068 | N/A | 33 | 21 | 3 |
| CIS-072 | N/A | 40 | 181.2 | 3 |
| CIS-074 | N/A | 37 | 518 | 3 |
| B006-249 | Cisplatin/Ifosfamide | 27 | ND* | 4 |
| CIS-002 | Cisplatin | 24 | 100.2 | 4 |
| CIS-025 | Cisplatin | 26 | 170.5 | 4 |
| CIS-028 | Cisplatin | 26 | 178 | 5 |
| CIS-034 | Cisplatin | 24 | 7.34 | 5 |
| CIS-043 | Cisplatin/Ifosfamide | 30 | 42 | 5 |
| CIS-033 | Cisplatin | 25 | 21 | 6 |
| CIS-044 | Cisplatin | 19 | 49 | 6 |
| CIS-046 | Cisplatin | 24 | 55 | 6 |

*ND indicates not determined

TABLE 2
Patient Information. Average and range for the chemotherapy treated
patients and controls presented for age at collection, age at treatment,
cisplatin dose (milligrams/meter squared), ifosfamide dose and
seminal fluid volume. Note that one patient that got 120 mg/m2
dose of cisplatin also got 800 mg/m2 of carboplatin which is in the
same class of drugs as cisplatin. The B006 chemotherapy case
sperm count was not determined (ND).

| | Chemotherapy Cases | Controls |
|---|---|---|
| Average (Mean) Age at | 24.78 | 36.01 |
| sample | | |
| Age Range at Sample | 19.12 to 29.86 | 27.5 to 44.4 |
| Average (Mean) Age at | 16.3 | NA |
| Treatment | | |
| Age Range at Treatment | 14.47 to 19.63 | NA |
| Average Cisplatin Dose | 389.33 mg/m² | NA |
| mg/m² | | |
| Cisplatin Dose Range mg/m² | 120* mg/m² to 480 mg/m² | NA |
| Average Ifosfamide Dose | 30.5 mg/m² | NA |
| mg/m² | | |
| Range Ifosfamide Dose | 30.5 mg/m² to 30.5 mg/m² | NA |
| mg/m² | | |
| Seminal Fluid Volume - | 2.23 ml | 2.41 ml |
| Average | | |
| Seminal Fluid Volume - | 0.6 to 5.0 | 0.3 to 7.5 |
| Range | | |
| # individuals with | 6 out of 9 | 6 out of 9 |
| Normal SF parameters | | |

NA indicates not applicable

DNA from the semen samples was isolated and equal amounts of DNA from 3 individuals pooled to generate three different pools of control (no previous chemotherapy) and three different pools of adolescent chemotherapy exposed cancer survivors labeled human sperm pools (HS#) #1 through #6. The pooled DNA was immunoprecipitated with 5 methyl cytosine monoclonal antibody for a methylated DNA immunoprecipitation (MeDIP). The MeDIP DNA was used to generate libraries and bar coded (index primers) separately for analysis by next generation sequencing (MeDIPSeq).

A high read number and alignment proportion was obtained (Table 3). The differential DNA methylation regions (DMRs) were identified using the MEDIPS R package as outlined in the Methods. The DMRs include single sites as well as multiple sites. The DMRs for all sites and multiple sites are shown for a variety of statistical pvalue thresholds in Table 4. The p<10⁻⁴ was selected for further analysis and due to the potential for false positives in single sites the multiple site p<10⁻⁴ was used for subsequent analysis and discussion. The more variable single sites are likely important, but the more stringently selected multiple site DMRs are used to convey the general observations. Therefore, the 2831 single sites and 135 multiple sites are discussed. The distribution of the DMR according to number of multiple sites is presented in Table 5 and the list of DMRs is shown in Table 6. The majority were single site DMRs with the bulk of the multiple site DMR having two sites. Interestingly, one DMR (DMR3:198096901) had 73 multiple windows on chromosome 3, none of which were associated with a known gene (Table 6). Observations demonstrate the adolescent chemotherapy exposure induced reproducible human sperm epimutations.

TABLE 3
DMR number and characteristics. The number of reads present for each
sample pool (HS #) and overall alignment rate calculated by bowtie2.
Read number and alignment

| | HS1 | HS2 | HS3 | HS4 | HS5 | HS6 |
|---|---|---|---|---|---|---|
| Read Number | 127916479 | 116715381 | 60181475 | 67042941 | 78944306 | 126030851 |
| Alignment % | 97.90 | 97.77 | 97.77 | 97.92 | 96.19 | 97.19 |

TABLE 4
DMR number and characteristics. The number of DMRs found using
different edgeR p-value cutoff thresholds.
DMR number

| p-value | Total DMR | Multiple Site DMR |
|---|---|---|
| 0.001 | 20526 | 1551 |
| 1.00E−04 | 2831 | 135 |
| 1.00E−05 | 463 | 24 |
| 1.00E−06 | 76 | 6 |
| 1.00E−07 | 15 | 2 |

TABLE 5
DMR number and characteristics. The number of DMR with associated
specific number of significant sites at a p-value threshold of <10⁻⁴.
Site number associated with DMR

| Number of Significant Sites | 1 | 2 | 3 | 4 | 5 | 6 | 8 | 73 |
|---|---|---|---|---|---|---|---|---|
| Number of DMR | 2696 | 110 | 13 | 5 | 4 | 1 | 1 | 1 |

TABLE 6
Human sperm chemotherapy-associated DMR list for multiple site DMR at p < 10⁻⁴. The DMR name,
chromosomal location, start site, length in base pair (bp), CpG density (CpG/100bp), and size
associated is listed. The absence (not applicable, NA) of one or more gene listed under “Gene
Association” indicates an intergenic DMR location.
Multiple Site DMR List

| | | | | | | | CpG | |
|---|---|---|---|---|---|---|---|---|
| | | | Length | | min P | CpG | Density | Gene |
| DMR Name | Chr | Start | (bp) | Windows | Value | # | (#/100bp) | Association |
| DMR1:12173801 | 1 | 12173801 | 1400 | 2 | 4.08E−05 | 27 | 1.9 | TNFRSF1B |
| DMR1:14192301 | 1 | 14192301 | 900 | 2 | 5.38E−05 | 6 | 0.6 | |
| DMR1:104846301 | 1 | 104846301 | 2500 | 2 | 5.56E−05 | 26 | 1 | |
| DMR1:154878801 | 1 | 154878801 | 700 | 2 | 1.08E−05 | 22 | 3.1 | |
| DMR1:175491101 | 1 | 175491101 | 3200 | 2 | 1.49E−05 | 47 | 1.4 | TNR |
| DMR1:215393901 | 1 | 215393901 | 500 | 2 | 6.42E−05 | 26 | 5.2 | RP11-199H2.2 |
| DMR1:224010101 | 1 | 224010101 | 4700 | 2 | 9.49E−06 | 108 | 2.2 | RP11-504P24.3 |
| DMR1:238863501 | 1 | 238863501 | 2100 | 2 | 1.33E−07 | 8 | 0.3 | |
| DMR2:2189101 | 2 | 2189101 | 1100 | 4 | 6.30E−07 | 35 | 3.1 | MYT1L |
| DMR2:35752601 | 2 | 35752601 | 4600 | 2 | 8.10E−06 | 24 | 0.5 | |
| DMR2:95550301 | 2 | 95550301 | 1300 | 2 | 3.14E−06 | 29 | 2.2 | |
| DMR2:144492201 | 2 | 144492201 | 1400 | 2 | 1.52E−06 | 11 | 0.7 | ZEB2 |
| DMR2:238198201 | 2 | 238198201 | 1100 | 2 | 3.28E−06 | 8 | 0.7 | ILKAP |
| DMR3:44495901 | 3 | 44495901 | 1400 | 2 | 4.11E−05 | 21 | 1.5 | |
| DMR3:55470001 | 3 | 55470001 | 1000 | 2 | 7.95E−07 | 35 | 3.5 | WNT5A |
| DMR3:113198301 | 3 | 113198301 | 200 | 2 | 4.33E−05 | 2 | 1 | |
| DMR3:198096901 | 3 | 198096901 | 10900 | 73 | 9.27E−30 | 397 | 3.6 | |
| DMR4:31437301 | 4 | 31437301 | 2600 | 2 | 9.28E−06 | 51 | 1.9 | |
| DMR4:146592201 | 4 | 146592201 | 400 | 2 | 1.71E−05 | 2 | 0.5 | |
| DMR4:186476501 | 4 | 186476501 | 1900 | 2 | 8.09E−06 | 17 | 0.8 | F11-AS1; |
| | | | | | | | | RP11-215A19. [symbol] |
| DMR4:188443001 | 4 | 188443001 | 6600 | 2 | 1.70E−06 | 99 | 1.5 | LINC01060 |
| DMR5:164501 | 5 | 164501 | 2200 | 3 | 8.79E−06 | 142 | 6.4 | PLEKHG4B |
| DMR5:561701 | 5 | 561701 | 3900 | 3 | 1.46E−07 | 396 | 10 | |
| DMR5:3879101 | 5 | 3879101 | 2700 | 2 | 6.99E−05 | 32 | 1.1 | |
| DMR5:4311001 | 5 | 4311001 | 700 | 3 | 9.20E−06 | 19 | 2.7 | |
| DMR5:9122501 | 5 | 9122501 | 700 | 2 | 1.21E−05 | 27 | 3.8 | SEMA5A |
| DMR5:23303701 | 5 | 23303701 | 1700 | 2 | 7.07E−05 | 44 | 2.5 | CTD-2272G21. [symbol] |
| DMR5:30068101 | 5 | 30068101 | 2900 | 2 | 1.28E−07 | 38 | 1.3 | |
| DMR5:55530601 | 5 | 55530601 | 500 | 2 | 2.89E−05 | 28 | 5.6 | PPAP2A; |
| | | | | | | | | RNF138P1 |
| DMR5:57549501 | 5 | 57549501 | 1400 | 2 | 1.42E−05 | 15 | 1 | |
| DMR5:77912901 | 5 | 77912901 | 1300 | 2 | 3.39E−05 | 24 | 1.8 | |
| DMR5:134314001 | 5 | 134314001 | 900 | 2 | 3.23E−05 | 10 | 1.1 | CTD-2410N18. [symbol] |
| | | | | | | | | CDKL3 |
| DMR5:151531101 | 5 | 151531101 | 1000 | 3 | 2.76E−06 | 44 | 4.4 | FAT2 |
| DMR5:162993101 | 5 | 162993101 | 800 | 2 | 1.10E−06 | 10 | 1.2 | |
| DMR6:1514701 | 6 | 1514701 | 1100 | 2 | 1.70E−05 | 38 | 3.4 | RP11-157J24.1 |
| DMR6:5092901 | 6 | 5092901 | 300 | 2 | 4.79E−05 | 2 | 0.6 | |
| DMR6:5792601 | 6 | 5792601 | 2400 | 2 | 1.74E−05 | 23 | 0.9 | |
| DMR6:31326001 | 6 | 31326001 | 1000 | 2 | 3.38E−05 | 17 | 1.7 | HLA-C |
| DMR6:31814701 | 6 | 31814701 | 3700 | 3 | 1.17E−05 | 217 | 5.8 | HSPA1L; |
| | | | | | | | | HSPA1A |
| DMR6:31828001 | 6 | 31828001 | 1600 | 2 | 2.62E−05 | 130 | 8.1 | HSPA1B |
| DMR6:58560701 | 6 | 58560701 | 2100 | 2 | 4.49E−06 | 37 | 1.7 | |
| DMR6:58593201 | 6 | 58593201 | 3200 | 2 | 6.04E−09 | 60 | 1.8 | |
| DMR6:59033701 | 6 | 59033701 | 600 | 2 | 3.44E−11 | 13 | 2.1 | |
| DMR6:59163101 | 6 | 59163101 | 1600 | 2 | 2.73E−06 | 34 | 2.1 | |
| DMR6:59274801 | 6 | 59274801 | 3200 | 2 | 1.30E−07 | 57 | 1.7 | |
| DMR6:59342901 | 6 | 59342901 | 1500 | 4 | 3.08E−06 | 36 | 2.4 | |
| DMR6:59565501 | 6 | 59565501 | 1000 | 2 | 5.85E−06 | 12 | 1.2 | |
| DMR6:59688201 | 6 | 59688201 | 200 | 2 | 7.98E−08 | 5 | 2.5 | |
| DMR6:116616001 | 6 | 116616001 | 300 | 2 | 3.05E−06 | 10 | 3.3 | |
| DMR6:167702601 | 6 | 167702601 | 2900 | 2 | 4.71E−05 | 70 | 2.4 | |
| DMR7:49018601 | 7 | 49018601 | 700 | 3 | 9.21E−07 | 14 | 2 | |
| DMR7:77441801 | 7 | 77441801 | 400 | 2 | 9.98E−07 | 6 | 1.5 | |
| DMR7:87205701 | 7 | 87205701 | 2300 | 2 | 3.39E−05 | 37 | 1.6 | TMEM243 |
| DMR7:101239401 | 7 | 101239401 | 1600 | 2 | 6.96E−06 | 66 | 4.1 | FIS1 |
| DMR7:109351301 | 7 | 109351301 | 400 | 2 | 1.94E−05 | 9 | 2.2 | |
| DMR7:158556901 | 7 | 158556901 | 1100 | 2 | 5.39E−06 | 65 | 5.9 | PTPRN2 |
| DMR8:7556101 | 8 | 7556101 | 500 | 2 | 8.30E−06 | 25 | 5 | |
| DMR8:27797501 | 8 | 27797501 | 500 | 2 | 5.39E−05 | 3 | 0.6 | ESCO2 |
| DMR8:44360401 | 8 | 44360401 | 300 | 2 | 3.67E−05 | 6 | 2 | |
| DMR8:45815901 | 8 | 45815901 | 300 | 2 | 9.64E−09 | 7 | 2.3 | |
| DMR8:45927501 | 8 | 45927501 | 6800 | 2 | 3.41E−06 | 122 | 1.7 | |
| DMR8:99694501 | 8 | 99694501 | 3100 | 3 | 5.63E−06 | 68 | 2.1 | VPS13B; |
| | | | | | | | | AC018442.1 |
| DMR8:126213701 | 8 | 126213701 | 300 | 2 | 1.09E−05 | 2 | 0.6 | |
| DMR8:142938801 | 8 | 142938801 | 1400 | 2 | 3.03E−05 | 55 | 3.9 | |
| DMR9:28333101 | 9 | 28333101 | 1100 | 2 | 3.09E−06 | 8 | 0.7 | LINGO2 |
| DMR9:40951901 | 9 | 40951901 | 500 | 2 | 4.31E−05 | 16 | 3.2 | |
| DMR9:41424201 | 9 | 41424201 | 600 | 2 | 7.72E−07 | 5 | 0.8 | |
| DMR9:95044801 | 9 | 95044801 | 1100 | 2 | 5.16E−07 | 50 | 4.5 | NPEPO |
| DMR9:98644001 | 9 | 98644001 | 1100 | 2 | 1.78E−05 | 12 | 1 | GABBR2 |
| DMR10:1197701 | 10 | 1197701 | 3100 | 8 | 1.51E−09 | 70 | 2.2 | ADARB2 |
| DMR10:15012201 | 10 | 15012201 | 1700 | 2 | 8.22E−05 | 42 | 2.4 | |
| DMR10:30846501 | 10 | 30846501 | 2900 | 2 | 6.76E−05 | 40 | 1.3 | ZNF438 |
| DMR10:32731701 | 10 | 32731701 | 400 | 2 | 3.00E−05 | 14 | 3.5 | CCDC7 |
| DMR10:73117601 | 10 | 73117601 | 300 | 2 | 1.77E−05 | 1 | 0.3 | NUDT13 |
| DMR10:90990401 | 10 | 90990401 | 400 | 2 | 8.86E−05 | 4 | 1 | |
| DMR10:123691901 | 10 | 123691901 | 800 | 2 | 1.57E−05 | 4 | 0.5 | GPR26 |
| DMR10:127538001 | 10 | 127538001 | 1800 | 2 | 2.69E−05 | 28 | 1.5 | |
| DMR10:129343401 | 10 | 129343401 | 2100 | 3 | 6.38E−06 | 49 | 2.3 | |
| DMR10:130440901 | 10 | 130440901 | 1100 | 2 | 2.10E−05 | 29 | 2.6 | RP11-540N6.1 |
| DMR11:484301 | 11 | 484301 | 4300 | 2 | 2.10E−06 | 312 | 7.2 | PTDSS2 |
| DMR11:47036401 | 11 | 47036401 | 1700 | 2 | 8.45E−06 | 33 | 1.9 | C11orf49 |
| DMR11:95437501 | 11 | 95437501 | 400 | 2 | 1.72E−05 | 8 | 2 | |
| DMR12:81062701 | 12 | 81062701 | 400 | 2 | 4.28E−07 | 2 | 0.5 | ACSS3 |
| DMR12:95948901 | 12 | 95948901 | 300 | 2 | 1.85E−05 | 3 | 1 | AMDHD1 |
| DMR12:130657401 | 12 | 130657401 | 2600 | 2 | 1.48E−05 | 80 | 3 | RP11-662M24. [symbol] |
| DMR13:63935201 | 13 | 63935201 | 300 | 2 | 8.12E−05 | 3 | 1 | |
| DMR13:98815001 | 13 | 98815001 | 3000 | 2 | 6.61E−05 | 41 | 1.3 | DOCK9 |
| DMR13:104088001 | 13 | 104088001 | 500 | 2 | 2.27E−07 | 7 | 1.4 | |
| DMR14:19433401 | 14 | 19433401 | 3600 | 3 | 2.54E−06 | 137 | 3.8 | POTEG |
| DMR14:38737701 | 14 | 38737701 | 300 | 2 | 1.02E−06 | 5 | 1.6 | |
| DMR14:46935601 | 14 | 46935601 | 300 | 2 | 5.76E−06 | 2 | 0.6 | MDGA2 |
| DMR14:62802301 | 14 | 62802301 | 500 | 2 | 1.46E−07 | 8 | 1.6 | KCNH5 |
| DMR15:20756701 | 15 | 20756701 | 2500 | 2 | 4.04E−05 | 46 | 1.8 | |
| DMR15:21172601 | 15 | 21172601 | 3800 | 4 | 4.00E−07 | 119 | 3.1 | |
| DMR15:21325001 | 15 | 21325001 | 5800 | 2 | 1.57E−06 | 135 | 2.3 | RP11-32B5.7; |
| | | | | | | | | RP11-275E15.2 |
| DMR15:73775201 | 15 | 73775201 | 300 | 2 | 3.11E−06 | 6 | 2 | |
| DMR15:88274701 | 15 | 88274701 | 300 | 2 | 7.18E−05 | 4 | 1.3 | |
| DMR15:88930701 | 15 | 88930701 | 400 | 3 | 9.67E−09 | 1 | 0.2 | |
| DMR16:2603101 | 16 | 2603101 | 1600 | 5 | 6.45E−07 | 77 | 4.8 | AC141586.5; |
| | | | | | | | | PDPK1 |
| DMR16:14910901 | 16 | 14910901 | 2800 | 5 | 9.51E−06 | 217 | 7.7 | MIR3180-1; |
| | | | | | | | | NPIPA3 |
| DMR16:61463701 | 16 | 61463701 | 1200 | 2 | 2.76E−06 | 2 | 0.1 | |
| DMR16:85972901 | 16 | 85972901 | 1200 | 2 | 3.25E−05 | 95 | 7.9 | |
| DMR17:121701 | 17 | 121701 | 6300 | 2 | 1.17E−05 | 115 | 1.8 | |
| DMR17:8836901 | 17 | 8836901 | 2500 | 2 | 3.82E−06 | 53 | 2.1 | PIK3R6 |
| DMR17:46382701 | 17 | 46382701 | 1600 | 2 | 1.94E−05 | 14 | 0.8 | NSFP1 |
| DMR17:68151901 | 17 | 68151901 | 900 | 2 | 4.81E−05 | 30 | 3.3 | LRRC37A16P |
| DMR18:8634901 | 18 | 8634901 | 200 | 2 | 1.21E−05 | 1 | 0.5 | RAB12 |
| DMR18:14484901 | 18 | 14484901 | 5100 | 2 | 1.18E−05 | 181 | 3.5 | GRAMD4P7; |
| | | | | | | | | CXADRP3 |
| DMR18:46969701 | 18 | 46969701 | 200 | 2 | 2.87E−05 | 18 | 9 | KATNAL2; |
| | | | | | | | | TCEB3CL |
| DMR18:59752001 | 18 | 59752001 | 300 | 2 | 1.46E−06 | 3 | 1 | |
| DMR18:65609101 | 18 | 65609101 | 600 | 2 | 2.08E−05 | 19 | 3.1 | RP11-775G23.1 |
| DMR18:70848101 | 18 | 70848101 | 900 | 2 | 1.62E−05 | 13 | 1.4 | |
| DMR18:76003301 | 18 | 76003301 | 2800 | 2 | 2.92E−07 | 39 | 1.3 | |
| DMR19:756801 | 19 | 756801 | 1600 | 2 | 3.50E−06 | 76 | 4.7 | MISP |
| DMR19:37842601 | 19 | 37842601 | 2500 | 2 | 1.14E−05 | 36 | 1.4 | AC016582.2 |
| DMR19:43206901 | 19 | 43206901 | 400 | 2 | 2.88E−06 | 16 | 4 | PSG4 |
| DMR19:48181401 | 19 | 48181401 | 1600 | 2 | 7.54E−06 | 51 | 3.1 | CARD8; |
| | | | | | | | | ZNF114; C19 |
| DMR19:52916701 | 19 | 52916701 | 4200 | 5 | 3.66E−10 | 101 | 2.4 | ZNF888 |
| DMR19:54772001 | 19 | 54772001 | 6000 | 4 | 8.20E−06 | 62 | 1 | KIR2DL1; |
| | | | | | | | | KIR3DL1: CT [symbol] |
| DMR20:2311001 | 20 | 2311001 | 800 | 2 | 7.56E−06 | 7 | 0.8 | TGM3 |
| DMR20:61964901 | 20 | 61964901 | 1500 | 2 | 8.95E−08 | 82 | 5.4 | TAF4 |
| DMR20:64131901 | 20 | 64131901 | 2700 | 3 | 1.68E−08 | 15 | 0.5 | |
| DMR21:7916001 | 21 | 7916001 | 26500 | 5 | 1.05E−06 | 710 | 2.6 | |
| DMR21:10652801 | 21 | 10652801 | 38000 | 3 | 9.10E−06 | 829 | 2.1 | |
| DMR21:42955701 | 21 | 42955701 | 200 | 2 | 2.98E−05 | 12 | 6 | |
| DMR21:44158201 | 21 | 44158201 | 1800 | 2 | 1.96E−05 | 51 | 2.8 | AP001055.6 |
| DMR22:11248401 | 22 | 11248401 | 8500 | 2 | 1.87E−05 | 383 | 4.5 | 5_8S_rRNA; |
| | | | | | | | | AC137488.1 |
| DMR22:32203601 | 22 | 32203601 | 3800 | 2 | 3.66E−06 | 159 | 4.1 | RP1-90G24.10 |
| DMR22:48701801 | 22 | 48701801 | 3100 | 2 | 3.52E−05 | 92 | 2.9 | FAM19A5 |
| DMRX:666401 | X | 666401 | 1000 | 2 | 1.66E−05 | 18 | 1.8 | |
| DMRX:1041801 | X | 1041801 | 9200 | 4 | 1.75E−06 | 164 | 1.7 | |
| DMRX:1235401 | X | 1235401 | 1100 | 2 | 1.72E−05 | 98 | 8.9 | |
| DMRX:3865201 | X | 3865201 | 3100 | 2 | 8.41E−05 | 83 | 2.6 | RP11-706O15. [symbol] |
| DMRX:115191101 | X | 115191101 | 1500 | 6 | 5.42E−07 | 128 | 8.5 | LRCH2; |
| | | | | | | | | RBMXL3 |
| DMRY:11559901 | Y | 11559901 | 33000 | 3 | 9.16E−06 | 910 | 2.7 | |

[symbol]

indicates data missing or illegible when filed

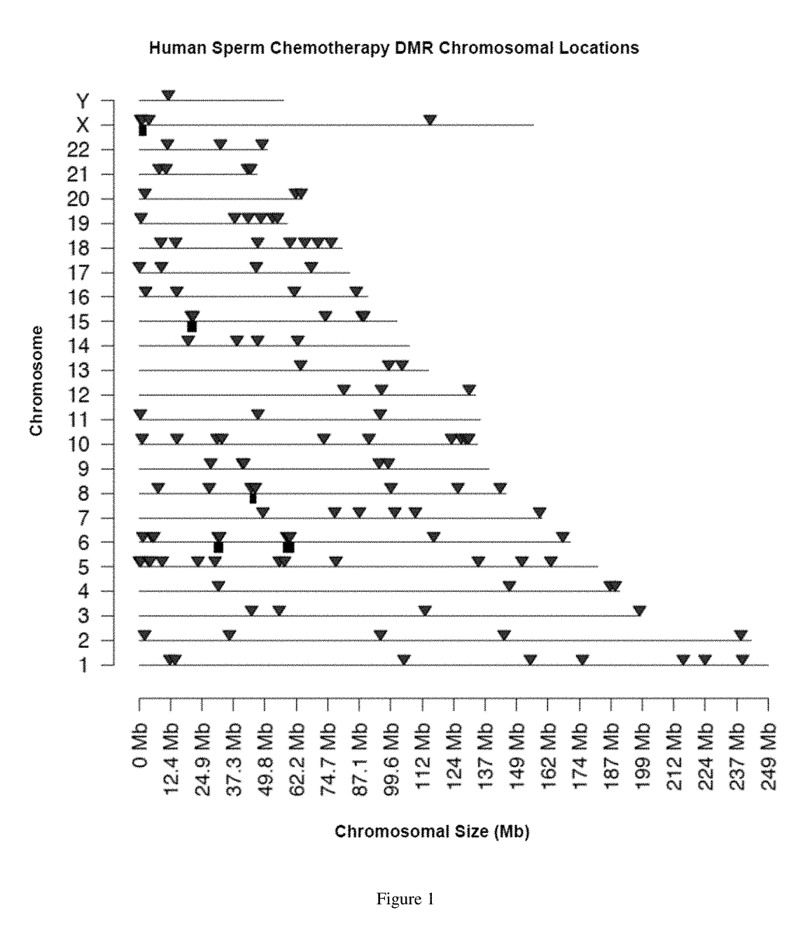

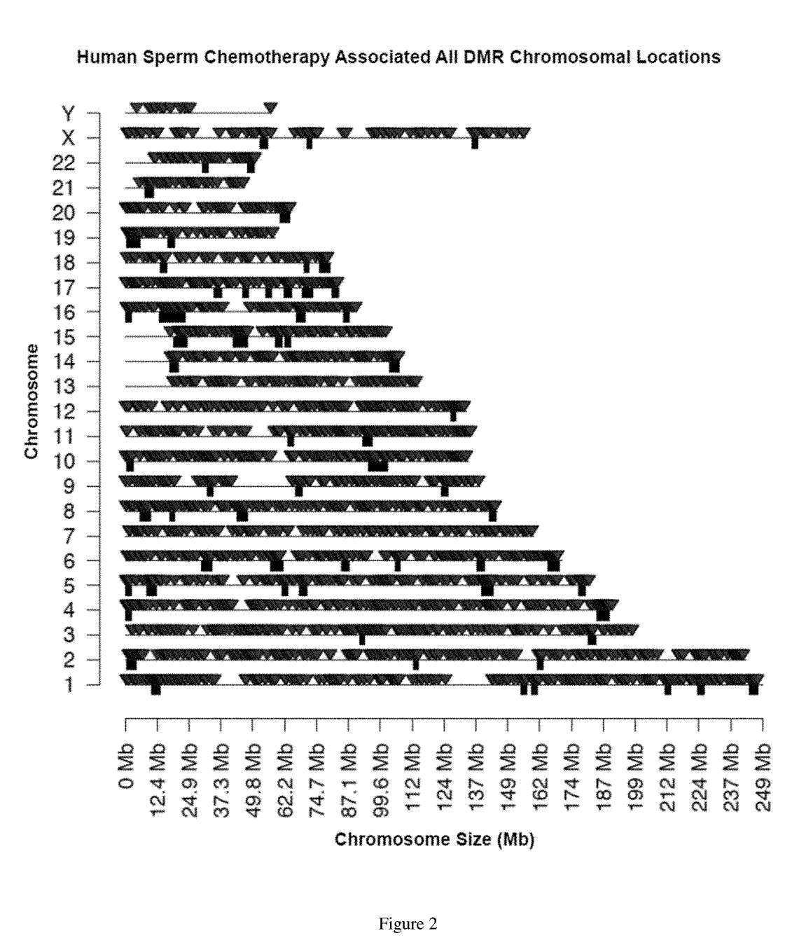

The chromosomal location of the sperm DMRs/epimutations is presented in FIG. 1. The DMRs were present on all chromosomes with a number of statistically over-represented clusters of DMRs indicated with the black box below the line. This epimutation signature was reproducible between the different human sperm pools. As a comparison the chromosomal plot of the single site DMRs is presented in FIG. 2. The single site DMR density was greater, but interestingly several regions in chromosome 1, 9, 13, 14, 15 were void of DMRs. In addition a larger number of DMR clusters were observed in all chromosomes (FIG. 2). The list of p<10⁻⁴ multiple site DMR is presented in Table 6. The current study demonstrates a signature of statistically significant epimutations that are present in the adolescent chemotherapy-exposure population. There may also be differential effects between different chemotherapies and periods of developmental exposure.

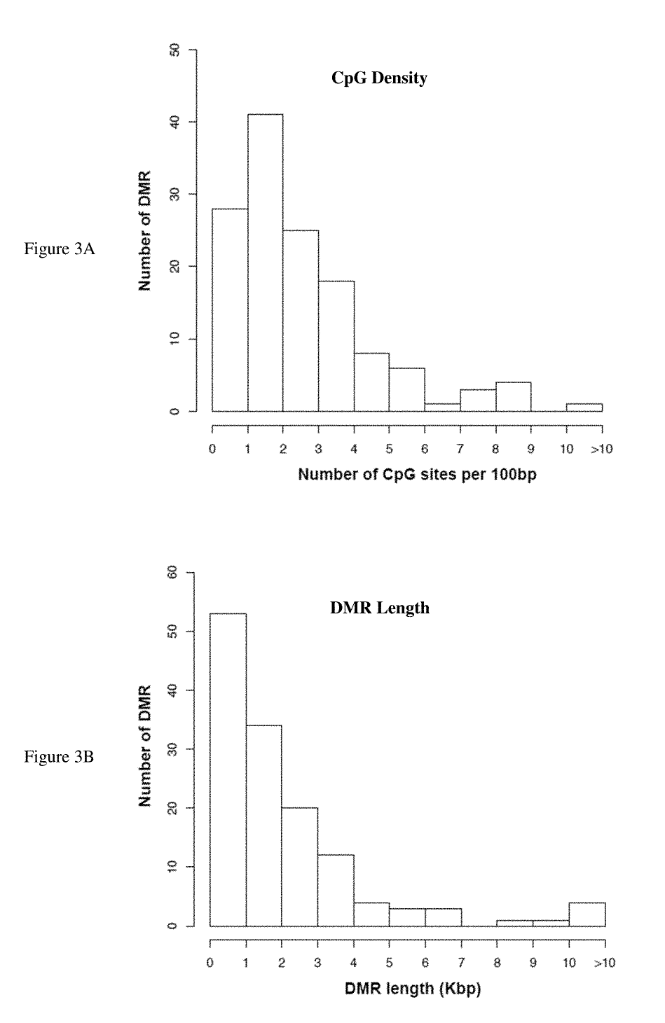

A genomic feature identified in all previously detected environmentally induced epimutations was a region of low density CpG content termed a CpG desert [20]. Analysis of the CpG content of the chemotherapy-associated human sperm epimutations identified between 1-3 CpG per 100 bp density with only one DMR having a greater than 10 CpG/100 bp (FIG. 3A and Table 6). Therefore, a CpG desert was a genomic feature of the chemotherapy-associated sperm DMRs. The epimutations were predominantly 1 kb in size with only a few greater than 6 kb in size (FIG. 3B and Table 6). Therefore, the genomic features of the human sperm epimutations identified were similar to those previously identified in other species [20].

The potential that molecular variation within each of the study populations may contribute to the DMR identified was investigated. Analysis of the internal population variation in the unexposed and exposed populations separately identified 114 and 50 single site DMR respectively. The three individual pools of each population were compared between each other to identify the internal population variation in DMR. The majority of internal population variation is anticipated to be hypervariable DMR, termed metastable epialleles [21], and none of these internal population DMR overlapped with the exposed versus unexposed DMR dataset. Therefore, internal population variation does not account for the chemotherapy associated DMR identified in sperm.

Analysis of a genetic mutation (copy number variation, CNV) was performed to determine the genetic CNV variation in the exposed versus unexposed comparison. Only 3 CNV were detected in the comparison. Although variable CNV were detected within the different pools of the populations, Table 7, comparison of the exposed versus unexposed populations identified minimal alterations present in all pool comparisons. None of the CNV were associated or overlapped with the DMR identified. Therefore, genetic CNV variation does not appear to be a cause for the epigenetic differences observed.

TABLE 7
CNV analysis summary for the human sperm. The nonexposed (HS1, HS2, HS3) control and chemotherapy exposed (HS4,
HS5, HS6) population pools are listed. Overlapping CNV between control and chemotherapy exposed population: 3 CNV.
CNV analysis summary for the human sperm
Read Mapping Summary

| | HS1 | HS2 | HS3 | HS4 | HS5 | HS6 |
|---|---|---|---|---|---|---|
| Read Number | 32352587 | 27963740 | 32524539 | 22628164 | 30272531 | 36013163 |
| Overall Alignment Rate | 97.41% | 78.89% | 96.76% | 93.03% | 86.87% | 94.47% |

The number of reads present for each sample and the overall alignment rate calculated by bowtie2.

Overall CNV Numbers:

| | 1 | 10 | 11 | 12 | 13 | 14 | 16 | 2 | 20 | 21 | 3 | 4 |
|---|---|---|---|---|---|---|---|---|---|---|---|---|
| HS1 | 7 | 1 | 14 | 4 | 3 | 7 | 2 | 4 | 4 | 5 | 7 | 8 |
| HS2 | 84 | 24 | 57 | 61 | 44 | 44 | 35 | 68 | 20 | 23 | 61 | 30 |
| HS3 | 110 | 48 | 35 | 22 | 26 | 34 | 41 | 77 | 24 | 18 | 65 | 50 |
| HS4 | 3 | 1 | 1 | 0 | 2 | 0 | 5 | 0 | 3 | 3 | 1 | 1 |
| HS5 | 5 | 3 | 3 | 2 | 0 | 2 | 0 | 1 | 4 | 1 | 2 | 1 |
| HS6 | 10 | 4 | 15 | 6 | 3 | 7 | 12 | 5 | 5 | 5 | 6 | 8 |
| | 5 | 6 | 7 | 8 | 9 | X | 15 | 18 | 19 | 22 | Y | 17 |
| HS1 | 5 | 3 | 8 | 5 | 12 | 2 | 0 | 0 | 0 | 0 | 0 | 0 |
| HS2 | 54 | 34 | 57 | 38 | 44 | 10 | 36 | 14 | 2 | 25 | 8 | 0 |
| HS3 | 53 | 39 | 43 | 46 | 21 | 14 | 17 | 23 | 33 | 21 | 0 | 43 |
| HS4 | 6 | 0 | 0 | 2 | 1 | 0 | 0 | 0 | 12 | 1 | 0 | 4 |
| HS5 | 1 | 0 | 1 | 1 | 4 | 0 | 2 | 0 | 0 | 2 | 0 | 0 |
| HS6 | 7 | 3 | 13 | 6 | 9 | 1 | 0 | 0 | 0 | 2 | 0 | 4 |

The number of CNV found, separated by sample and chromosome.

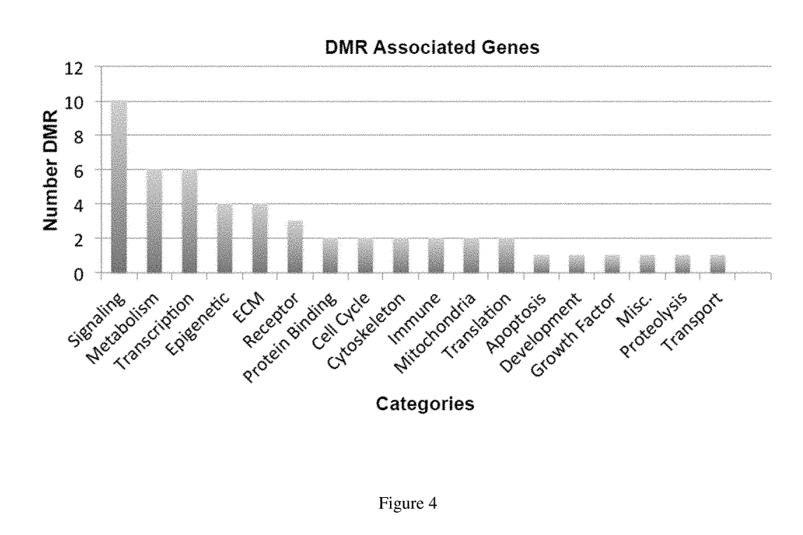

The gene associations with the DMRs are listed in Table 8 and the complete list with information in Tables 6 and 9. Approximately 50% of the DMRs had associations with genes indicating half the epimutations are intergenic and distal from genes. Previously some DMRs have been suggested to potentially act as epigenetic control regions and distally regulate expression through ncRNA mechanisms for 2-5 Mbase regions [22]. The genes associated with chemotherapy-associated DMRs are present in numerous gene classifications with no major category being overrepresented (Tables 8 and 9). The number of DMR associated with specific gene classification categories are presented in FIG. 4. The DMR associated genes were analyzed for correlated known gene pathways. No specific pathway or cellular process was found to have more than four associated genes. These results suggest that the chemotherapy induced sperm DMR can alter genome activity.

TABLE 8
DMR associated gene list and categories. The specific DMR,
associated gene symbol and classification category are
presented. Some DMR are associated with multiple genes
which are listed. DMR associated genes with unknown
classification only are not listed.

| DMR Name | Gene Association | Category |
|---|---|---|
| DMR2:238198201 | ILKAP | Signaling |
| DMR5:164501 | PLEKHG4B | Signaling |
| DMR5:55530601 | PPAP2A | Signaling |
| | RNF138P1 | Unknown |
| DMR5:9122501 | SEMA5A | Signaling |
| DMR7:158556901 | PTPRN2 | Signaling |
| DMR8:99694501 | VPS13B | Signaling |
| | AC018442.1 | Unknown |
| DMR13:98815001 | DOCK9 | Signaling |
| DMR16:2603101 | PDPK1 | Signaling |
| | AC141586.5 | Unknown |
| DMR17:8836901 | PIK3R6 | Signaling |
| DMR18:8634901 | RAB12 | Signaling |
| DMR10:73117601 | NUDT13 | Metabolism |
| DMR11:484301 | PTDSS2 | Metabolism |
| DMR12:81062701 | ACSS3 | Metabolism |
| DMR12:95948901 | AMDHD1 | Metabolism |
| DMR14:46935601 | MDGA2 | Metabolism |
| DMR20:2311001 | TGM3 | Metabolism |
| DMR2:2189101 | MYT1L | Transcription |
| DMR2:144492201 | ZEB2 | Transcription |
| DMR10:30846501 | ZNF438 | Transcription |
| DMR10:32731701 | CCDC7 | Transcription |
| DMR19:52916701 | ZNF888 | Transcription |
| DMR20:61964901 | TAF4 | Transcription |
| DMR4:186476501 | F11-AS1 | Epigenetic |
| | RP11-215A19.2 | Unknown |
| DMR4:188443001 | LINC01060 | Epigenetic |
| DMR8:27797501 | ESCO2 | Epigenetic |
| DMR16:14910901 | MIR3180-1 | Epigenetic |
| | NPIPA3 | Unknown |
| | RP11-958N24.1 | Unknown |
| | NPIPA1 | Unknown |
| DMR1:175491101 | TNR | ECM |
| DMR5:151531101 | FAT2 | ECM |
| DMR9:28333101 | LINGO2 | ECM |
| DMR19:43206901 | PSG4 | ECM |
| DMR1:12173801 | TNFRSF1B | Receptor |
| DMR9:98644001 | GABBR2 | Receptor |
| DMR10:123691901 | GPR26 | Receptor |
| DMR6:31814701 | HSPA1L | Protein |
| | HSPA1A | Binding |
| | | Protein |
| | | Binding |
| DMR6:31828001 | HSPA1B | Protein |
| | | Binding |
| DMR5:134314001 | CDKL3 | Cell Cycle |
| | CTD-2410N18.4 | Unknown |
| DMR19:756801 | MISP | Cell Cycle |
| DMR18:46969701 | KATNAL2 | Cytoskeleton |
| | TCEB3CL | Transcription |
| DMRX:115191101 | LRCH2 | Cytoskeleton |
| | RBMXL3 | Translation |
| DMR6:31326001 | HLA-C | Immune |
| DMR19:54772001 | KIR2DL1 | Immune |
| | KIR3DL1 | Immune |
| | CTB-61M7.1 | Unknown |
| DMR7:87205701 | TMEM243 | Mitochondria |
| DMR7:101239401 | FIS1 | Mitochondria |
| DMR10:1197701 | ADARB2 | Translation |
| DMR22:11248401 | 5_8S_rRNA | Translation |
| | AC137488.1 | Unknown |
| DMR19:48181401 | CARD8 | Apoptosis |
| | ZNF114 | Transcription |
| | C19orf68 | Unknown |
| DMR3:55470001 | WNT5A | Development |
| DMR22:48701801 | FAM19A5 | Growth |
| | | Factor |
| DMR12:130657401 | RIMBP2 | Misc. |
| | RP11-662M24.2 | Unknown |
| | RP11-662M24.1 | Unknown |
| DMR9:95044801 | NPEPO | Proteolysis |
| DMR14:62802301 | KCNH5 | Transport |

TABLE 9
Human sperm chemotherapy-associated DMR associated genes. The DMR name, gene symbol, chromosome location
start and end position, Ensembl number, gene description and classification category are presented.

| | | Multiple Site DMR Associated Genes | | | | | |
|---|---|---|---|---|---|---|---|
| | | | start | end | | | Classification |
| DMR Name | Gene Symbol | Chr | position | position | Ensembl # | Gene Description | Category |
| DMR1:12173801 | TNFRSF1B | 1 | 12167003 | 12209228 | ENSG00000028137 | tumor necrosis | Receptor |
| | | | | | | factor receptor | |
| | | | | | | superfamily - | |
| | | | | | | member 1B | |
| DMR1:175491101 | TNR | 1 | 175315194 | 175743770 | ENSG00000116147 | tenascin R | ECM |
| DMR1:215393901 | RP11- | 1 | 215393646 | 215394418 | ENSG00000282265 | NA | Unknown |
| | 199H2.2 | | | | | | |
| DMR1:224010101 | RP11- | 1 | 223992743 | 224010612 | ENSG00000185495 | NA | Unknown |
| | 504P24.3 | | | | | | |
| DMR2:2189101 | MYT1L | 2 | 1789113 | 2331260 | ENSG00000186487 | myelin | Transcription |
| | | | | | | transcription | |
| | | | | | | factor 1-like | |
| DMR2:144492201 | ZEB2 | 2 | 144384081 | 144524583 | ENSG00000169554 | zinc finger E-box | Transcription |
| | | | | | | binding homeobox | |
| | | | | | | 2 | |
| DMR2:238198201 | ILKAP | 2 | 238170401 | 238203729 | ENSG00000132323 | integrin-linked | Signaling |
| | | | | | | kinase-associated | |
| | | | | | | serine/threonine | |
| | | | | | | phosphatase | |
| DMR3:55470001 | WNT5A | 3 | 55465715 | 55490539 | ENSG00000114251 | wingless-type | Development |
| | | | | | | MMTV | |
| | | | | | | integration site | |
| | | | | | | family - member | |
| | | | | | | 5A | |
| DMR4:186476501 | F11-AS1 | 4 | 186286094 | 186500997 | ENSG00000251165 | F11 antisense | Epigenetic |
| | | | | | | RNA 1 | |
| DMR4:186476501 | RP11- | 4 | 186426546 | 186555328 | ENSG00000272297 | Uncharacterized | Unknown |
| | 215A19.2 | | | | | protein | |
| DMR4:188443001 | LINC01060 | 4 | 188400736 | 188681051 | ENSG00000249378 | long intergenic | Epigenetic |
| | | | | | | non-protein | |
| | | | | | | coding RNA 1060 | |
| DMR5:164501 | PLEKHG4B | 5 | 140258 | 189970 | ENSG00000153404 | pleckstrin | Signaling |
| | | | | | | homology domain | |
| | | | | | | containing - | |
| | | | | | | family G (with | |
| | | | | | | RhoGef domain) | |
| | | | | | | member 4B | |
| DMR5:9122501 | SEMA5A | 5 | 9035026 | 9546075 | ENSG00000112902 | sema domain - | Signaling |
| | | | | | | seven | |
| | | | | | | thrombospondin | |
| | | | | | | repeats (type 1 and | |
| | | | | | | type 1-like) - | |
| | | | | | | transmembrane | |
| | | | | | | domain (TM) | |
| DMR5:23303701 | CTD- | 5 | 23303565 | 23305143 | ENSG00000250332 | NA | Unknown |
| | 2272G21.2 | | | | | | |
| DMR5:55530601 | PPAP2A | 5 | 55424854 | 55535050 | ENSG00000067113 | phosphatidic acid | Signaling |
| | | | | | | phosphatase type | |
| | | | | | | 2A | |
| DMR5:55530601 | RNF138P1 | 5 | 55530156 | 55530701 | ENSG00000250853 | ring finger protein | Unknown |
| | | | | | | 138 - E3 ubiquitin | |
| | | | | | | protein ligase | |
| | | | | | | pseudogene 1 | |
| DMR5:134314001 | CTD- | 5 | 134205614 | 134371044 | ENSG00000273345 | NA | Unknown |
| | 2410N18.4 | | | | | | |
| DMR5:134314001 | CDKL3 | 5 | 134286350 | 134371047 | ENSG00000006837 | cyclin-dependent | Cell Cycle |
| | | | | | | kinase-like 3 | |
| DMR5:151531101 | FAT2 | 5 | 151504093 | 151568944 | ENSG00000086570 | FAT atypical | ECM |
| | | | | | | cadherin 2 | |
| DMR6:1514701 | RP11- | 6 | 1513698 | 1515289 | ENSG00000218027 | NA | Unknown |
| | 157J24.1 | | | | | | |
| DMR6:31326001 | HLA-C | 6 | 31268749 | 31357158 | ENSG00000204525 | major | Immune |
| | | | | | | histocompatibility | |
| | | | | | | complex - class I - | |
| | | | | | | C | |
| DMR6:31814701 | HSPA1L | 6 | 31809619 | 31815065 | ENSG00000204390 | heat shock 70 kDa | Protein Binding |
| | | | | | | protein 1-like | |
| DMR6:31814701 | HSPA1A | 6 | 31815464 | 31817946 | ENSG00000204389 | heat shock 70 kDa | Protein Binding |
| | | | | | | protein 1A | |
| DMR6:31828001 | HSPA1B | 6 | 31827735 | 31830255 | ENSG00000204388 | heat shock 70 kDa | Protein Binding |
| | | | | | | protein 1B | |
| DMR7:87205701 | TMEM243 | 7 | 87196160 | 87220587 | ENSG00000135185 | transmembrane | Mitochondria |
| | | | | | | protein 243 - | |
| | | | | | | mitochondrial | |
| DMR7:101239401 | FIS1 | 7 | 101239458 | 101252316 | ENSG00000214253 | fission 1 | Mitochondria |
| | | | | | | (mitochondrial | |
| | | | | | | outer membrane) | |
| | | | | | | homolog | |
| | | | | | | (S. cerevisiae) | |
| DMR7:158556901 | PTPRN2 | 7 | 157539056 | 158587788 | ENSG00000155093 | protein tyrosine | Signaling |
| | | | | | | phosphatase - | |
| | | | | | | receptor type - N | |
| | | | | | | polypeptide 2 | |
| DMR8:27797501 | ESCO2 | 8 | 27771949 | 27812640 | ENSG00000171320 | establishment of | Epigenetic |
| | | | | | | sister chromatid | |
| | | | | | | cohesion N- | |
| | | | | | | acetyltransferase 2 | |
| DMR8:99694501 | VPS13B | 8 | 99013266 | 99877580 | ENSG00000132549 | vacuolar protein | Signaling |
| | | | | | | sorting 13 | |
| | | | | | | homolog B (yeast) | |
| DMR8:99694501 | AC018442.1 | 8 | 99695957 | 99698017 | ENSG00000235683 | NA | Unknown |
| DMR9:28333101 | LINGO2 | 9 | 27948078 | 28670286 | ENSG00000174482 | leucine rich repeat | ECM |
| | | | | | | and Ig domain | |
| | | | | | | containing 2 | |
| DMR9:95044801 | NPEPO | 9 | 94726701 | 95087218 | ENSG00000148120 | chromosome 9 | Proteolysis |
| | | | | | | open reading | |
| | | | | | | frame 3 | |
| DMR9:98644001 | GABBR2 | 9 | 98288109 | 98709197 | ENSG00000136928 | gamma- | Receptor |
| | | | | | | aminobutyric acid | |
| | | | | | | (GABA) B | |
| | | | | | | receptor - 2 | |
| DMR10:1197701 | ADARB2 | 10 | 1177318 | 1737476 | ENSG00000185736 | adenosine | Translation |
| | | | | | | deaminase - RNA- | |
| | | | | | | specific - B2 (non- | |
| | | | | | | functional) | |
| DMR10:30846501 | ZNF438 | 10 | 30820207 | 31031937 | ENSG00000183621 | zinc finger protein | Transcription |
| | | | | | | 438 | |
| DMR10:32731701 | CCDC7 | 10 | 32567723 | 32882874 | ENSG00000150076 | coiled-coil domain | Transcription |
| | | | | | | containing 7 | |
| DMR10:73117601 | NUDT13 | 10 | 73110375 | 73131828 | ENSG00000166321 | nudix (nucleoside | Metabolism |
| | | | | | | diphosphate linked | |
| | | | | | | moiety X)-type | |
| | | | | | | motif 13 | |
| DMR10:123691901 | GPR26 | 10 | 123666355 | 123694607 | ENSG00000154478 | G protein-coupled | Receptor |
| | | | | | | receptor 26 | |
| DMR10:130440901 | RP11- | 10 | 130439067 | 130483154 | ENSG00000236303 | NA | Unknown |
| | 540N6.1 | | | | | | |
| DMR11:484301 | PTDSS2 | 11 | 448268 | 491399 | ENSG00000174915 | phosphatidylserine | Metabolism |
| | | | | | | synthase 2 | |
| DMR11:47036401 | C11orf49 | 11 | 46936689 | 47164385 | ENSG00000149179 | chromosome 11 | Unknown |
| | | | | | | open reading | |
| | | | | | | frame 49 | |
| DMR12:81062701 | ACSS3 | 12 | 80936414 | 81261205 | ENSG00000111058 | acyl-CoA | Metabolism |
| | | | | | | synthetase short- | |
| | | | | | | chain family | |
| | | | | | | member 3 | |
| DMR12:95948901 | AMDHD1 | 12 | 95943293 | 95968716 | ENSG00000139344 | amidohydrolase | Metabolism |
| | | | | | | domain containing | |
| | | | | | | 1 | |
| DMR12:130657401 | RP11- | 12 | 130628316 | 130716281 | ENSG00000256725 | NA | Unknown |
| | 662M24.2 | | | | | | |
| DMR12:130657401 | RIMBP2 | 12 | 130396137 | 130716281 | ENSG00000060709 | RIMS binding | Misc. |
| | | | | | | protein 2 | |
| DMR12:130657401 | RP11- | 12 | 130651371 | 130669233 | ENSG00000256343 | NA | Unknown |
| | 662M24.1 | | | | | | |
| DMR13:98815001 | DOCK9 | 13 | 98793487 | 99086625 | ENSG00000088387 | dedicator of | Signaling |
| | | | | | | cytokinesis 9 | |
| DMR14:19433401 | POTEG | 14 | 19402486 | 19434341 | ENSG00000187537 | POTE ankyrin | Unknown |
| | | | | | | domain family - | |
| | | | | | | member G | |
| DMR14:46935601 | MDGA2 | 14 | 46839629 | 47674954 | ENSG00000139915 | MAM domain | Metabolism |
| | | | | | | containing | |
| | | | | | | glycosylphosphati- | |
| | | | | | | dylinositol anchor | |
| | | | | | | 2 | |
| DMR14:62802301 | KCNH5 | 14 | 62699454 | 63102037 | ENSG00000140015 | potassium channel - | Transport |
| | | | | | | voltage gated eag | |
| | | | | | | related subfamily | |
| | | | | | | H - member 5 | |
| DMR15:21325001 | RP11-32B5.7 | 15 | 21298233 | 21325241 | ENSG00000247765 | NA | Unknown |
| DMR15:21325001 | RP11- | 15 | 21328380 | 21343881 | ENSG00000280881 | NA | Unknown |
| | 275E15.2 | | | | | | |
| DMR16:2603101 | AC141586.5 | 16 | 2603350 | 2630494 | ENSG00000215154 | NA | Unknown |
| DMR16:2603101 | PDPK1 | 16 | 2537964 | 2603188 | ENSG00000140992 | 3- | Signaling |
| | | | | | | phosphoinositide | |
| | | | | | | dependent protein | |
| | | | | | | kinase 1 | |
| DMR16:14910901 | MIR3180-1 | 16 | 14911220 | 14911313 | ENSG00000265537 | microRNA 3180-1 | Epigenetic |
| DMR16:14910901 | NPIPA3 | 16 | 14708944 | 14952073 | ENSG00000224712 | nuclear pore | Unknown |
| | | | | | | complex | |
| | | | | | | interacting protein | |
| | | | | | | family - member | |
| | | | | | | A3 | |
| DMR16:14910901 | RP11- | 16 | 14911551 | 14935708 | ENSG00000183458 | NA | Unknown |
| | 958N24.1 | | | | | | |
| DMR16:14910901 | NPIPA1 | 16 | 14750813 | 14952060 | ENSG00000183426 | nuclear pore | Unknown |
| | | | | | | complex | |
| | | | | | | interacting protein | |
| | | | | | | family - member | |
| | | | | | | A1 | |
| DMR17:8836901 | PIK3R6 | 17 | 8802723 | 8867677 | ENSG00000276231 | phosphoinositide- | Signaling |
| | | | | | | 3-kinase - | |
| | | | | | | regulatory subunit | |
| | | | | | | 6 | |
| DMR17:46382701 | NSFP1 | 17 | 46372855 | 46487141 | ENSG00000260075 | N-ethylmaleimide- | Unknown |
| | | | | | | sensitive factor | |
| | | | | | | pseudogene 1 | |
| DMR17:68151901 | LRRC37A16P | 17 | 68125777 | 68152468 | ENSG00000267023 | leucine rich repeat | Unknown |
| | | | | | | containing 37 - | |
| | | | | | | member A16 - | |
| | | | | | | pseudogene | |
| DMR17:68151901 | RP11- | 17 | 68152776 | 68159043 | ENSG00000267708 | NA | Unknown |
| | 147L13.7 | | | | | | |
| DMR18:8634901 | RAB12 | 18 | 8609445 | 8639381 | ENSG00000206418 | RAB12 - member | Signaling |
| | | | | | | RAS oncogene | |
| | | | | | | family | |
| DMR18:14484901 | GRAMD4P7 | 18 | 14485806 | 14487501 | ENSG00000266242 | GRAM domain | Unknown |
| | | | | | | containing 4 | |
| | | | | | | pseudogene 7 | |
| DMR18:14484901 | CXADRP3 | 18 | 14477955 | 14499278 | ENSG00000265766 | coxsackie virus | Unknown |
| | | | | | | and adenovirus | |
| | | | | | | receptor | |
| | | | | | | pseudogene 3 | |
| DMR18:46969701 | KATNAL2 | 18 | 46917492 | 47102243 | ENSG00000167216 | katanin p60 | Cytoskeleton |
| | | | | | | subunit A-like 2 | |
| DMR18:46969701 | TCEB3CL | 18 | 46968695 | 47029842 | ENSG00000275553 | transcription | Transcription |
| | | | | | | elongation factor | |
| | | | | | | B polypeptide 3C- | |
| | | | | | | like | |
| DMR18:65609101 | RP11- | 18 | 65606090 | 65652053 | ENSG00000265217 | NA | Unknown |
| | 775G23.1 | | | | | | |
| DMR19:756801 | MISP | 19 | 751126 | 764318 | ENSG00000099812 | mitotic spindle | Cell Cycle |
| | | | | | | positioning | |
| DMR19:37842601 | AC016582.2 | 19 | 37823722 | 37855215 | ENSG00000225868 | NA | Unknown |
| DMR19:43206901 | PSG4 | 19 | 43192702 | 43207299 | ENSG00000243137 | pregnancy specific | ECM |
| | | | | | | beta-1- | |
| | | | | | | glycoprotein 4 | |
| DMR19:48181401 | CARD8 | 19 | 48180770 | 48255946 | ENSG00000105483 | caspase | Apoptosis |
| | | | | | | recruitment | |
| | | | | | | domain family - | |
| | | | | | | member 8 | |
| DMR19:48181401 | ZNF114 | 19 | 48172318 | 48287608 | ENSG00000178150 | zinc finger protein | Transcription |
| | | | | | | 114 | |
| DMR19:48181401 | C19orf68 | 19 | 48170692 | 48197620 | ENSG00000185453 | chromosome 19 | Unknown |
| | | | | | | open reading | |
| | | | | | | frame 68 | |
| DMR19:52916701 | ZNF888 | 19 | 52915196 | 52923470 | ENSG00000213793 | zinc finger protein | Transcription |
| | | | | | | 888 | |
| DMR19:54772001 | KIR2DL1 | 19 | 54769811 | 54784322 | ENSG00000125498 | killer cell | Immune |
| | | | | | | immunoglobulin- | |
| | | | | | | like receptor - two | |
| | | | | | | domains - long | |
| | | | | | | cytoplasmic tail - | |
| | | | | | | 1 | |
| DMR19:54772001 | KIR3DL1 | 19 | 54724497 | 54867215 | ENSG00000167633 | killer cell | Immune |
| | | | | | | immunoglobulin- | |
| | | | | | | like receptor - | |
| | | | | | | three domains - | |
| | | | | | | long cytoplasmic | |
| | | | | | | tail - 1 | |
| DMR19:54772001 | CTB-61M7.1 | 19 | 54724496 | 54798285 | ENSG00000215765 | | Unknown |
| DMR20:2311001 | TGM3 | 20 | 2296001 | 2341078 | ENSG00000125780 | transglutaminase 3 | Metabolism |
| | | | | | | [Source: HGNC | |
| | | | | | | Symbol; Acc: | |
| | | | | | | HGNC: 11779] | |
| DMR20:61964901 | TAF4 | 20 | 61953469 | 62065810 | ENSG00000130699 | TAF4 RNA | Transcription |
| | | | | | | polymerase II - | |
| | | | | | | TATA box | |
| | | | | | | binding protein | |
| | | | | | | (TBP)-associated | |
| | | | | | | factor - 135 kDa | |
| DMR21:44158201 | AP001055.6 | 21 | 44158740 | 44160076 | ENSG00000225331 | NA | Unknown |
| DMR22:11248401 | 5_8S_rRNA | 22 | 11249809 | 11249959 | ENSG00000276871 | 5.8S ribosomal | Translation |
| | | | | | | RNA | |
| | | | | | | [Source: RFAM; | |
| | | | | | | Acc: RF00002] | |
| DMR22:11248401 | AC137488.1 | 22 | 11253605 | 11253719 | ENSG00000277683 | | Unknown |
| DMR22:32203601 | RP1- | 22 | 32205115 | 32269666 | ENSG00000242082 | NA | Unknown |
| | 90G24.10 | | | | | | |
| DMR22:48701801 | FAM19A5 | 22 | 48489460 | 48850912 | ENSG00000219438 | family with | Growth Factor |
| | | | | | | sequence | |
| | | | | | | similarity 19 | |
| | | | | | | (chemokine (C-C | |
| | | | | | | motif)-like) - | |
| | | | | | | member A5 | |
| DMRX:3865201 | RP11- | X | 3853010 | 3882317 | ENSG00000234449 | NA | Unknown |
| | 706O15.3 | | | | | | |
| DMRX:115191101 | LRCH2 | X | 115110616 | 115234072 | ENSG00000130224 | leucine-rich | Cytoskeleton |
| | | | | | | repeats and | |
| | | | | | | calponin | |
| | | | | | | homology (CH) | |
| | | | | | | domain containing | |
| | | | | | | 2 | |
| DMRX:115191101 | RBMXL3 | X | 115189427 | 115192868 | ENSG00000175718 | RNA binding | Translation |
| | | | | | | motif protein - X- | |
| | | | | | | linked-like 3 | |

Table 10 (see associated .txt file). Human sperm DMR for all DMR sites, single and multiple, at a p-value threshold of 1e-04. The DMR name, chromosome location, start and stop base pair location, length in base pair (bp), number of significant windows (100 bp), p-value, number of CpG sites, CpG sites per 100 bp, and DMR associated gene symbol (annotation) are provided.

### Discussion

The observations described herein suggest that altered sperm DNA methylation may result from early life cancer chemotherapy exposure and correlate to alterations in sperm morphology, number and ultimately male fertility. Although other epigenetic changes could also be involved, DNA methylation has been shown to have more developmental and genome wide influences than many of the other epigenetic factors [23]. This study is the first examination of the actions of current chemotherapy regimens on the human sperm epigenome and spermatogenesis. Previous studies have suggested no evidence in humans of adverse effects of chemotherapy treatment in offspring (less than five years of age) of male cancer survivors [24-26]. However, at the time of these studies, many male survivors had not yet attempted to sire a pregnancy and the number of pregnancies from partners of male survivors were small. No studies have examined later life adult generational impacts.

Previous studies in non-human model systems have demonstrated that a variety of exposures can promote epigenetic alterations in the germline [6, 7, 14, 27, 28]. Environmental toxicants including the fungicide vinclozolin [7, 8], pesticides DDT and methoxychlor [7], plastic derived compounds BPA and phthalates [15], and hydrocarbons [16] can promote altered epigenetic (DNA methylation) programming in sperm [14]. The ability of environmental exposures to promote sperm epimutations suggested that chemotherapy may also promote altered germ cell epigenetic programming. The current study was designed to investigate the effects of adolescent chemotherapy exposure on later life adult sperm epimutations. The results demonstrate the presence of DMRs or epimutations in the sperm of men that had adolescent chemotherapy exposure.

Approximately a decade had passed since the cancer patients' chemotherapy. More advanced spermatogenic cells would have been lost after 100 days following chemotherapy due to the developmental period of the spermatogenic cells in the testis. The observation of epigenetic alterations in the sperm long after chemotherapy strongly suggests that the spermatogonial stem cells in the testis had a permanent epigenetic alteration such that the adult male will produce sperm with epimutations throughout life.

The analysis and selection under high stringency (i.e. multiple site DMR with p<10⁻⁴) identified a group (i.e. signature) of sperm epimutations associated with chemotherapy exposed individuals. The lower stringency single site DMRs identified are more variable between individuals, but also reflect chemotherapy exposure associated DMR. The presence of a significant epimutation chemotherapy signature demonstrates the ability of early life chemotherapy to promote germline epimutations.

The current study was designed to examine adolescent (i.e. pubertal) male exposure, however, since the same populations of spermatogenic stem cells are present throughout adult life, potential chemotherapy induced sperm epimutations may occur any time a male is exposed to chemotherapy. Therefore, the cryopreservation of gametes prior to chemotherapy may be important for patients and their oncologists to consider in the future [4].

The sperm epimutations identified were present on all chromosomes with a number being clustered in statistically significant over-represented groups of DMR. The clustering of DMR is speculated to represent critical regulatory regions within epigenetic control regions [22]. Interestingly, the genomic features of these human sperm chemotherapy associated epigenetics were similar to previously identified sperm epimutations. In particular, one of the major genomic features is a low density CpG content within the DMR referred to as a CpG desert [20]. The CpG density was less than ten percent and the mean was around two CpG/100 bp. Due to the evolutionary conservation of these CpG clusters in a CpG desert they are speculated to be regulatory sites [20].

The selection of DMR was focused on multiple site DMR with a high statistical significance. Although a higher rate of false positives is anticipated in the much more common single site DMRs, these single sites are anticipated to be an important component of the chemotherapy induced sperm epimutations. Expanded studies are needed to further investigate the epimutation profiles in the sperm and the physiological impacts. The degree of internal population DMR variation and genetic CNV variation indicated negligible impact on the DMR detected. A large proportion of the epimutations identified were found to have gene associations. No predominant pathways or cellular processes appear over-represented by the epimutation associated genes. Previous studies have demonstrated the ability of DMR/epimutations to cause altered somatic cell gene expression [22]. Therefore transmission of the sperm epimutations to the subsequent generation may alter somatic cell gene activity in offspring.

The germline (e.g. sperm) transmission of epigenetic information can promote the epigenetic transgenerational inheritance of disease and phenotypic variation [12, 13, 22]. A variety of environmental factors from nutrition to toxicants have been shown in a variety of species from plants to humans to promote the epigenetic transgenerational inheritance phenomenon [6]. Since epigenetic inheritance requires the germline (egg or sperm) transmission of epigenetic information between generations [6, 9-11, 27, 28], the alterations of epigenetic processes in the germline need to be established. Developmentally the DNA methylation is erased after fertilization to create the embryonic stem cell totipotency, which then is remethylated in a cell specific manner during embryonic development [29]. Therefore, the majority of the DNA methylation is reset upon fertilization and during primordial germ cell development of the germline [29, 30].

However, a set of genes termed imprinted genes are protected from DNA methylation erasure at fertilization allowing them to be transmitted transgenerationally [6]. In the event an environmental exposure modified the epigenetic programming of the germline (e.g. sperm) and these sites become imprinted-like they can promote the epigenetic transgenerational inheritance of disease [6, 31]. Previous studies have documented the ability of caloric restriction to induce the epigenetic inheritance of disease in humans [11, 32]. The current study identifies the ability of chemotherapy to reprogram the epigenome of the sperm. These epimutations can be transmitted to the developing embryo of the next generation. In the event these are imprinted-like epimutations then they would not be erased and would promote epigenetic inheritance to the next generation, and potentially epigenetic transgenerational inheritance to subsequent generations. The current study suggests that chemotherapy has the ability to induce epigenetic inheritance to subsequent generations.

In summary, the current study demonstrates for the first time the ability of chemotherapy to promote epigenetic reprogramming in the spermatogonial stem cell population that will lead to human sperm epimutations later in life. These DMRs have some gene associations that could influence genome activity. A highly reproducible set of epimutations (i.e. signature) was detected and may provide an epigenetic biomarker for chemotherapy exposures. The biological impact of chemotherapy induced epimutations may be to transmit altered epigenetic information to the next generation and if imprinted-like to subsequent generations progeny.

### Methods

### Study Population and Samples

The patients were 19-30 year-old male survivors of osteosarcoma recruited from the Seattle Children's Hospital in Seattle Wash. and four collaborating institutions (Children's Hospital of Pennsylvania, Philadelphia, Pa.; Miller Children's, Long Beach, Calif.; Children's Hospital, University of Minnesota, Minneapolis, Minn.; and Children's Hospital, Vanderbilt University, Nashville, Tenn.). These men had been treated for their disease with cisplatin-based chemotherapy regimens, including ifosfamide in some cases, when they were 14-20 years of age. Each patient was recruited by in-clinic or mail recruitment protocols. Male survivors were eligible if they met the following criteria: alive, with no evidence of disease; diagnosed with bone or soft tissue sarcoma; off all cancer treatment, including radiation treatment, for at least 2 years; at least 15 years of age at study entry; less than 21 at diagnosis; had received cisplatin as part of cancer treatment; must not have received any other alkylating agent (Cyclophosphamide, Melphalan, Busulfan, BCNU, CCNU, Chlorambucil, Nitrogen Mustard, Procarabazine, or Thiotepa); must have received all or part of their cancer treatment at one of the collaborating sites; free of any pre-condition to cancer treatment that could result in infertility; have had no CNS, abdominal, pelvic, or gonadal radiation therapy or total body irradiation (TBI); proficiency in English as designated in patient's medical record; provided informed consent or assent, and authorization to access medical records under HIPAA. Note that relapsed patients and patients with a subsequent malignancy (SMN) that are treated with surgery alone for the relapse or SMN were eligible for this study as long as they meet the above criteria. Controls were recruited from among adult men with no history of cancer who had previously participated as controls in the Fred Hutchinson Cancer Research Center, Seattle Wash., ATLAS study [34, 35]. These men were re-contacted regarding participation in the current study.

Each patient and control was asked to provide a semen sample via home seminal fluid collection, which we used to allow for ease of subject participation since the sample can be obtained without the individual traveling to a laboratory. Sperm concentration and morphology measures were performed on semen that had undergone liquefication during shipping, consistent with the WHO protocol for semen analysis [36]. For sperm concentration (per ml), each participant's semen was diluted and assessed by CASA (Computer Assisted Sperm Analysis). Three separate counts were performed and the results averaged. A known volume of semen was washed for making smears for morphology assessments, based on 200 sperm. Although sperm motility was not assessed (because it requires a fresh sample), count and morphology data nonetheless provide a great deal of information regarding spermatogenesis and abnormalities and both are associated with an increased risk of infertility [37]. All protocols were approved by the Seattle Children's Hospital institutional IRB committee (#12839 and 13158).

### DNA Preparation

Frozen human sperm samples were stored at −20° C. and thawed for analysis. Genomic DNA from sperm was prepared as follows: One hundred μl of sperm suspension was used then 820 μl. DNA extraction buffer (50 mM Tris pH 8, 10 mM EDTA pH 8, 0.5% SDS) and 80 μl 0.1 M Dithiothreitol (DTT) added and the sample incubated at 65° C. for 15 minutes. Eighty μl Proteinase K (20 mg/ml) was added and the sample incubated on a rotator at 55° C. for 2 hours. After incubation, 300 μl of protein precipitation solution (Promega, A795A, Madison, Wis.) was added, the sample mixed and incubated on ice for 15 minutes, then spun at 4° C. at 13,000 rpm for 20 minutes. The supernatant was transferred to a fresh tube, then precipitated over night with the same volume 100% isopropanol and 2 μl glycoblue at −20° C. The sample was then centrifuged and the pellet washed with 75% ethanol, then air-dried and resuspended in 100 μl H2O. DNA concentration was measured using the Nanodrop (Thermo Fisher, Waltham, Mass.).

### Methylated DNA Immunoprecipitation MeDIP

Methylated DNA Immunoprecipitation (MeDIP) with genomic DNA was performed as follows: Human sperm DNA pools were generated using 2 μg of genomic DNA from each individual for 3 pools each of control and chemotherapy exposed subjects. Each pool contained 3 individuals for a total of n=9 per exposure group. The resulting 6 μg of genomic DNA per pool was diluted to 150 μl with 1× Tris-EDTA (TE, 10 mM Tris, 1 mM EDTA) and sonicated with a probe sonicator using 5×20 pulses at 20% amplitude.

Fragment size (200-800 bp) was verified on a 1.5% agarose gel. Sonicated DNA was diluted to 400 μl with 1×TE and heated to 95° C. for 10 minutes, then incubated in ice water for 10 minutes. Then 100μl of 5× immunoprecipitation (IP) buffer (50 mM Sodium Phosphate pH 7, 700 mM NaCl, 0.25% Triton X-100) and 5 μg of 5-mC monoclonal antibody (Diagenode, Denville, N.J., C15200006-500) were added and the sample incubated on a rotator at 4° C. over night. The next day Protein A/G Agarose Beads from Santa Cruz were prewashed with 1×PBS/0.1% BSA and resuspended in 1×IP buffer.

Eighty μl of the bead slurry were added to each sample and incubated at 4° C. for 2 hours on a rotator. The bead-DNA-antibody complex was washed 3 times with 1×IP buffer by centrifuging at 6,000 rpm for 2 minutes and resuspending in 1×IP buffer. After the last wash the bead-complex was resuspended in 250 μl of digestion buffer (50 mM Tris pH 8, 10 mM EDTA pH 8, 0.5% SDS) with 3.5 μl Proteinase K (20 mg/ml) per sample and incubated on a rotator at 55° C. for 2 hours. After incubation DNA was extracted with the same volume of Phenol-Chloroform-Isoamyalcohol and then with the same volume chloroform. To the supernatant from chloroform extraction 2 μl glycoblue, 20 μl 5M Sodium Chloride and 500 μl 100% cold ethanol were added. DNA was precipitated at −20° C. over night, then spun for 20 minutes at 13,000 rpm at 4 C, washed with 75% ethanol and air-dried. Dry pellet was resuspended in 20 μl H2O and concentration measured in Qubit using the Qubit ssDNA Assay Kit (Life Technologies, Carlsbad, Calif.).

### MeDIP-Seq Analysis

The MeDIP pools were used to create libraries for next generation sequencing (NGS) at the University of Reno, Nev. Genomics Core Laboratory using the NEBNEXT® ULTRA™ RNA Library Prep Kit for Illumina® (San Diego, Calif.) starting at step 1.4 of the manufacturer's protocol to generate double stranded DNA. After this step the manufacturer's protocol was followed. Each pool received a separate index primer. NGS was performed at that same laboratory using the Illumina® HiSeq 2500 with a PESO application, with a read size of approximately 50 bp and approximately 100 million reads per pool. Two libraries each were run in one lane comparing one control with one chemotherapy exposed pool in each lane.

### CNV-Seq Analysis

Genomic DNA extracted from sperm was used to create pools containing the same individuals as used for MeDIP-seq. Equal amounts of each individual's genomic DNA were used for each pool with a final amount of 2 μg per pool. The pools were diluted to 130 μl with 1×TE buffer and sonicated in a Covaris M220 with the manufacturer's preset program to create fragments with a peak at 300 bp. Aliquots of the pools were run on a 1.5% agarose gel to confirm fragmentation. The NEBNEXT® DNA Library Kit was used to create libraries for each pool, with each pool receiving a separate index primer. The libraries were sent to the WSU Genomics Core in Spokane, Wash. for NGS on the Illumina® HiSeq 2500 using a PESO application. All 6 libraries were run in one lane and comparisons were performed. Approximately 30 million reads were obtained for each sample for comparison.

### Bioinformatics and Statistics

Basic read quality was verified using summaries produced by the FastQC program [38]. The reads for each sample for both CNV and DMR analyses were mapped to the GRCh38 human genome using Bowtie2 [39] with default parameter options. The mapped read files were then converted to sorted BAM files using SAMtools [40]. To identify DMR, the reference genome was broken into 100 bp windows. The MEDIPS R package [41] was used to calculate differential coverage between control and exposure sample groups. The edgeR p-value [42] was used to determine the relative difference between the two groups for each genomic window. Windows with an edgeR p-value less than 10⁻⁴ were considered DMRs. The DMR edges were extended until no genomic window with an edgeR p-value less than 0.1 remained within 1000 bp of the DMR. CpG density and other information was then calculated for the DMR based on the reference genome. The DMRs that included at least two windows with an edgeR p-value <10⁻⁴ were then selected for further analysis and annotated.

The cn.MOPS R package [43] was used to identify potential CNV. The cn.MOPS analysis detects CNVs by modeling read depth across all samples. The window size used by the cn.MOPS analysis was chosen dynamically for each chromosome based on the read coverage. For chromosomes 1 to 22 the window size ranged from 10 kb to 20 kb. For the MT, X, and Y chromosomes the window sizes were 1 kb, 31 kb, and 42 kb, respectively. We considered only CNV that occurred exclusively in either all control or all treatment samples.

DMR clusters were identified with R script using a 2 Mb sliding window with 50 kb intervals. DMR were annotated using the biomaRt R package [44] to access the Ensembl database [45]. The genes that overlapped with DMR were then input into the KEGG pathway search [46, 47] to identify associated pathways. The DMR associated genes were manually then sorted into functional groups by consulting information provided by the DAVID [48], Panther [49], and Uniprot databases incorporated into an internal curated database.

### REFERENCES

1. Howlader, N., Noone, A. M. & Waldron, W. SEER Cancer Statistics Review, 1975-2008. (National Cancer Institute, Bethesda, Md., 2011).
2. Zebrack, B. J., Casillas, J., Nohr, L., Adams, H. & Zeltzer, L. K. Fertility issues for young adult survivors of childhood cancer. Psycho-oncology 13, 689-699 (2004).
3. Hammond, C., Abrams, J. R. & Syrjala, K. L. Fertility and risk factors for elevated infertility concern in 10-year hematopoietic cell transplant survivors and casematched controls. Journal of clinical oncology: official journal of the American Society of Clinical Oncology 25, 3511-3517 (2007).
4. Saito, K., Suzuki, K., Iwasaki, A., Yumura, Y. & Kubota, Y. Sperm cryopreservation before cancer chemotherapy helps in the emotional battle against cancer. Cancer 104, 521-524 (2005).
5. Schover, L. R. Psychosocial aspects of infertility and decisions about reproduction in young cancer survivors: a review. Medical and pediatric oncology 33, 53-59 (1999).
6. Skinner, M. K. Endocrine disruptor induction of epigenetic transgenerational inheritance of disease. Molecular and cellular endocrinology 398, 4-12 (2014).
7. Anway, M. D., Cupp, A. S., Uzumcu, M. & Skinner, M. K. Epigenetic transgenerational actions of endocrine disruptors and male fertility. Science 308, 1466-1469 (2005).
8. Guerrero-Bosagna, C., Settles, M., Lucker, B. & Skinner, M. Epigenetic transgenerational actions of vinclozolin on promoter regions of the sperm epigenome. PloS one 5, e13100 (2010).
9. Skinner, M. K. Environmental epigenetic transgenerational inheritance and somatic epigenetic mitotic stability. Epigenetics: official journal of the DNA Methylation Society 6, 838-842 (2011).
10. Gapp, K., von Ziegler, L., Tweedie-Cullen, R. Y. & Mansuy, I. M. Early life epigenetic programming and transmission of stress-induced traits in mammals: how and when can environmental factors influence traits and their transgenerational inheritance? BioEssays: news and reviews in molecular, cellular and developmental biology 36, 491-502 (2014).
11. Pembrey, M., Saffery, R. & Bygren, L. O. Human transgenerational responses to early-life experience: potential impact on development, health and biomedical research. J Med Genet 51, 563-572 (2014).
12. Nilsson, E., et al. Environmentally Induced Epigenetic Transgenerational Inheritance of Ovarian Disease. PloS one 7, e36129 (2012).
13. Guerrero-Bosagna, C., Savenkova, M., Haque, M. M., Sadler-Riggleman, I. & Skinner, M. K. Environmentally Induced Epigenetic Transgenerational Inheritance of Altered Sertoli Cell Transcriptome and Epigenome: Molecular Etiology of Male Infertility. PloS one 8, e59922 (2013).
14. Manikkam, M., Guerrero-Bosagna, C., Tracey, R., Haque, M. M. & Skinner, M. K. Transgenerational actions of environmental compounds on reproductive disease and identification of epigenetic biomarkers of ancestral exposures. PloS one 7, e31901 (2012).
15. Manikkam, M., Tracey, R., Guerrero-Bosagna, C. & Skinner, M. Plastics Derived Endocrine Disruptors (BPA, DEHP and DBP) Induce Epigenetic Transgenerational Inheritance of Adult-Onset Disease and Sperm Epimutations. PloS one 8, e55387 (2013).
16. Tracey, R., Manikkam, M., Guerrero-Bosagna, C. & Skinner, M. Hydrocarbons (jet fuel JP-8) induce epigenetic transgenerational inheritance of obesity, reproductive disease and sperm epimutations. Reproductive toxicology 36, 104-116 (2013).
17. Nilsson, E. E., Anway, M. D., Stanfield, J. & Skinner, M. K. Transgenerational epigenetic effects of the endocrine disruptor vinclozolin on pregnancies and female adult onset disease. Reproduction 135, 713-721 (2008).
18. Anway, M. D., Leathers, C. & Skinner, M. K. Endocrine disruptor vinclozolin induced epigenetic transgenerational adult-onset disease. Endocrinology 147, 5515-5523 (2006).
19. Aston, K. I., et al. Aberrant sperm DNA methylation predicts male fertility status and embryo quality. Fertility and sterility (2015).
20. Skinner, M. K. & Guerrero-Bosagna, C. Role of CpG Deserts in the Epigenetic Transgenerational Inheritance of Differential DNA Methylation Regions. BMC Genomics 15, 692 (2014).
21. Dominguez-Salas, P., et al. Maternal nutrition at conception modulates DNA methylation of human metastable epialleles. Nature communications 5, 3746 (2014).
22. Skinner, M. K., Manikkam, M., Haque, M. M., Zhang, B. & Savenkova, M. Epigenetic Transgenerational Inheritance of Somatic Transcriptomes and Epigenetic Control Regions. Genome biology 13, R91 (2012).
23. Jirtle, R. L. & Skinner, M. K. Environmental epigenomics and disease susceptibility. Nature reviews. Genetics 8, 253-262 (2007).
24. Green, D. M., et al. Pregnancy outcome after treatment for Wilms tumor: a report from the national Wilms tumor long-term follow-up study. J Clin Oncol 28, 2824-2830 (2010).
25. Hawkins, M. M. Is there evidence of a therapy-related increase in germ cell mutation among childhood cancer survivors? J Natl Cancer Inst 83, 1643-1650 (1991).
26. Byrne, J., et al. Genetic disease in offspring of long-term survivors of childhood and adolescent cancer. Am J Hum Genet 62, 45-52 (1998).
27. Kelly, W. G. Transgenerational epigenetics in the germline cycle of Caenorhabditis elegans. Epigenetics Chromatin 7, 6 (2014).
28. Gapp, K., et al. Implication of sperm RNAs in transgenerational inheritance of the effects of early trauma in mice. Nat Neurosci 17, 667-669 (2014).
29. Feng, S., Jacobsen, S. E. & Reik, W. Epigenetic reprogramming in plant and animal development. Science 330, 622-627 (2010).
30. Seisenberger, S., et al. The dynamics of genome-wide DNA methylation reprogramming in mouse primordial germ cells. Molecular cell 48, 849-862 (2012).
31. Barlow, D. P. & Bartolomei, M. S. Genomic imprinting in mammals. Cold Spring Harbor perspectives in biology 6 (2014).
32. Donkin, I., et al. Obesity and Bariatric Surgery Drive Epigenetic Variation of Spermatozoa in Humans. Cell metabolism (2015).
33. Wyrobek, A. J., Schmid, T. E. & Marchetti, F. Relative susceptibilities of male germ cells to genetic defects induced by cancer chemotherapies. Journal of the National Cancer Institute. Monographs, 31-35 (2005).
34. Littman, A. J., et al. Physical activity in adolescence and testicular germ cell cancer risk. Cancer causes & control: CCC 20, 1281-1290 (2009).
35. Daling, J. R., et al. Association of marijuana use and the incidence of testicular germ cell tumors. Cancer 115, 1215-1223 (2009).
36. World Health Organization WHO Manual for the Examination of Human Semen and Sperm-Cervical Mucus Interaction, (Cambridge University Press, NY, 1999).
37. Guzick, D. S., et al. Sperm morphology, motility, and concentration in fertile and infertile men. The New England journal of medicine 345, 1388-1393 (2001).
38. Andrews, S. FastQC: a quality control tool for high throughput sequence data.
39. Langmead, B. & Salzberg, S. L. Fast gapped-read alignment with Bowtie 2. Nature methods 9, 357-359 (2012).
40. Li, H., et al. The Sequence Alignment/Map format and SAMtools. Bioinformatics 25, 2078-2079 (2009).
41. Lienhard, M., Grimm, C., Morkel, M., Herwig, R. & Chavez, L. MEDIPS: genomewide differential coverage analysis of sequencing data derived from DNA enrichment experiments. Bioinformatics 30, 284-286 (2014).
42. Robinson, M. D., McCarthy, D. J. & Smyth, G. K. edgeR: a Bioconductor package for differential expression analysis of digital gene expression data. Bioinformatics 26, 139-140 (2010).
43. Klambauer, G., et al. cn.MOPS: mixture of Poissons for discovering copy number variations in next-generation sequencing data with a low false discovery rate. Nucleic acids research 40, e69 (2012).
44. Durinck, S., Spellman, P. T., Birney, E. & Huber, W. Mapping identifiers for the integration of genomic datasets with the R/Bioconductor package biomaRt. Nature protocols 4, 1184-1191 (2009).
45. Cunningham, F., et al. Ensembl 2015. Nucleic acids research 43, D662-669 (2015).
46. Kanehisa, M. & Goto, S. KEGG: kyoto encyclopedia of genes and genomes. Nucleic acids research 28, 27-30 (2000).
47. Kanehisa, M., et al. Data, information, knowledge and principle: back to metabolism in KEGG. Nucleic acids research 42, D199-205 (2014).
48. Huang da, W., Sherman, B. T. & Lempicki, R. A. Systematic and integrative analysis of large gene lists using DAVID bioinformatics resources. Nature protocols 4, 44-57 (2009).
49. Mi, H., Muruganujan, A., Casagrande, J. T. & Thomas, P. D. Large-scale gene function analysis with the PANTHER classification system. Nature protocols 8, 1551-1566 (2013).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of determining if a male subject has been exposed to a chemotherapy agent comprising
obtaining at least one genomic DNA sequence from a semen sample from said male subject;
identifying the presence or absence of an epigenetic modification at one or more regions of said at least one genomic DNA, wherein said epigenetic modification comprises at least one differential DNA methylation region (DMR) listed in Table 6 or Table 10; and
determining that said subject has been exposed to a chemotherapy agent if said epigenetic modification is identified to be present in said at least one genomic DNA sequence.

2. The method of claim 1, wherein said epigenetic modification comprises a plurality of DMRs selected from the group listed in Table 6 or Table 10.

3. The method of claim 1, wherein said epigenetic modification comprises each DMR listed in Table 6 or Table 10.

4. The method of claim 1, wherein said chemotherapy agent is at least one of cisplatin and ifosfamide.

5. A method of screening for pregnancy complications, infertility, and passage of heritable mutations to an infant attributable to a male subject that has previously undergone chemotherapy treatment comprising
obtaining at least one genomic DNA sequence from a semen sample from said male subject that has previously undergone chemotherapy treatment;
identifying the presence or absence of an epigenetic modification at one or more regions of said at least one genomic DNA, wherein said epigenetic modification comprises at least one DMR listed in Table 6 or Table 10; and
indicating that said subject is at high risk of infertility or of passing heritable mutations which can lead to pregnancy complications or mutations in an infant if said epigenetic modification is identified to be present in said at least one genomic DNA sequence.

6. The method of claim 5, wherein said epigenetic modification comprises a plurality of DMRs selected from the group listed in Table 6 or Table 10.

7. The method of claim 5, wherein said epigenetic modification comprises each DMR listed in Table 6 or Table 10.

8. The method of claim 5, wherein said male subject underwent chemotherapy treatment for a period of time at an age prior to reproduction.

9. The method of claim 5, wherein said male subject underwent chemotherapy treatment for a period of time at an age from 14 and 20 years old.

10. The method of claim 5, wherein said chemotherapy treatment comprised at least one of cisplatin and ifosfamide.

11. A method for the early intervention and treatment of a male subject who is suspected of or who has been exposed to chemotherapy treatment, comprising
obtaining at least one genomic DNA sequence from a semen sample from said male subject that has previously undergone chemotherapy treatment;
identifying the presence or absence of an epigenetic modification at one or more regions of said at least one genomic DNA, wherein said epigenetic modification comprises at least one DMR listed in Table 6 or Table 10;
indicating that said subject is at high risk of infertility or of passing heritable mutations which can lead to pregnancy complications or mutations in an infant if said epigenetic modification is identified to be present in said at least one genomic DNA sequence; and
administering an appropriate treatment protocol to said subject determined to be at high risk of infertility or of passing heritable mutations which can lead to pregnancy complications or mutations in an infant.
